(19) United States
(12) Patent Application Publication
(10) Pub. No.: US 2025/0282861 A1
(43) Pub. Date: Sep. 11, 2025

(54) MFAP4 AND TREATMENT OF FIBROSIS

(71) Applicant: SYDDANSK UNIVERSITET, Odense M (DK)

(72) Inventors: Grith LYKKE SØRENSEN, Odense S (DK); Anders SCHLOSSER, Odense SV (DK); Uffe HOLMSKOV, Odense S (DK)

(21) Appl. No.: 18/554,751

(22) PCT Filed: Apr. 11, 2022
(86) PCT No.: PCT/EP2022/059600

§ 371 Date: Oct. 10, 2023

(30) Foreign Application Priority Data
Apr. 12, 2021 (EP) 21167836.2
Feb. 3, 2022 (EP) 22155019.7

(51) Int. Cl. C07K 16/28; A61K 39/00; A61P 1/16; A61P 27/02

(52) U.S. Cl. CPC C07K 16/28; A61P 1/16; A61P 27/02; A61K 2039/505

(57) ABSTRACT

The present invention relates to a selective targeting agent, which is capable of binding MFAP4 and inhibiting MFAP4-integrin interaction for use in the prevention, alleviation and/or treatment of fibrosis. The invention further comprises compositions comprising said selective targeting agents.

### CROSS-REFERENCE TO RELATED APPLICATIONS

This application claims priority to co-pending PCT Application No. PCT/EP2022/059600 filed Apr. 11, 2022, which claims priority to European Application No. 22155019.7 filed on Feb. 3, 2022 and European Application No. 21167836.2 filed on Apr. 12, 2021, all of which are hereby incorporated by reference in their entirety.

### SEQUENCE LISTING

The instant application contains a Sequence Listing which has been submitted electronically as an ASCII text file and is hereby incorporated by reference in its entirety. Said ASCII text file, created on Jan. 24, 2024, is named Updated sequence list 16SYDD-PV10302PA.txt and is 14,275 bytes in size.

### TECHNICAL FIELD OF THE INVENTION

The present invention relates to the prevention, alleviation and/or treatment of fibrosis such as liver fibrosis. In particular, the present invention relates to the use of selective targeting agents against MFAP4 in the prevention, alleviation and/or treatment of fibrosis.

### BACKGROUND OF THE INVENTION

Fibrosis is characterized by excessive ECM deposition following repeated injury and is also known as fibrotic scarring as fibrosis is a critical part of the wound healing process. In the pathological wound healing process connective tissue replaces normal parenchymal tissue. Hence, fibrosis is an exaggerated wound healing response, which interferes with normal organ function. It includes but is not restricted to the replacement of resident cells by TGF−-activated myofibroblasts and collagen type I deposition. Overall, the tissue repair mechanism is rather complex with a tight regulation of the extracelluar matrix synthesis and degradation to ensure maintenance of normal tissue architecture. However, the wound healing response often becomes deregulated leading to an irreversible fibrotic response if tissue injury is severe or repetitive.

Fibrosis is commonly observed as causing several diseases and is important in disease pathogenesis as excessive ECM deposition often results in loss of tissue function. It is a leading cause of morbidity and mortality and may affect all tissues such as lung, skin, liver, kidney and heart. Accordingly, fibrosis may result in several severe diseases like liver cirrhosis, kidney diseases, cystic fibrosis, arthrofibrosis, idiopathic pulmonary fibrosis and hypertrophic cardiomyopathy.

There are limited or no treatment options of fibrosis. Direct inhibition of the TGF−-pathway has not translated to clinical application and unfavorable risk-benefit profiles have resulted in termination of trials. Therapeutic feasibility has been demonstrated for idiopathic pulmonary fibrosis (IPF) only, a deadly condition of unknown cause, with the approval of oral drugs Pirfenidone and Nintedanib. However, these drugs are associated with adverse gastrointestinal effects, provide no cure and do not reverse fibrosis.

The ECM is a complex network that is highly regulated by multiple pathways. One of the proteins, which participates in the organization of the ECM, is microfibrillar-associated protein 4 (MFAP4). The ECM-binding efficacy of MFAP4 facilitates the correct assembly of ECM-fibers including fibrillin and elastin and participates in the organization of ECM through direct interaction with elastin, fibrillin and collagen.

The role of MFAP4 in fibrosis has been studied in MFAP4-deficient mice showing contradictory results. MFAP4-deficiency have shown both no effect on the development of experimental liver fibrosis after CCl4 treatment or on development of experimental pulmonary fibrosis after pulmonary bleomycin installation as well as a decrease in fibronectin and collagen I deposition in unilateral ureteral obstruction. Also, for cardiac fibrosis the effect in MFAP4-deficient mice is somewhat contradictory, showing results of both lower levels of cardiac fibrosis and fewer ventricular arrhythmias as well as no difference in cardiac collagen deposition is observed in a different study. Thus, the interaction between MFAP4 and fibrosis appears to be complex given the contradictory results.

Accordingly, the complexity of the ECM influences the balance between fibrolysis and fibrogenesis and may also to some extent influence and counter-balance one other. If the balance is shifted this may lead to fibrosis but no compounds exist today for reversing this imbalance in order to treat fibrosis. Hence, treatment of fibrosis by reversing the excessive deposition of ECM in the tissue to allow normal parenchymal tissue to regenerate and avoid dysfunction of the tissues is highly demanded.

### SUMMARY OF THE INVENTION

The present invention provides efficient and reliable selective targeting agents such as antibodies and fragments hereof and a composition comprising the selective targeting agents for treating, alleviating and/or preventing fibrosis. These selective targeting agents are capable of targeting MFAP4 and furthermore to de-differentiate myofibroblasts and hereby reduce and/or reverse the synthesis of collagen.

Thus, an object of the present invention relates to the provision of compounds and compositions comprising these compounds for treating, alleviating and/or preventing fibrosis.

Thus, one aspect of the invention relates to a selective targeting agent, which is capable of binding MFAP4 and inhibiting MFAP4-integrin interaction for use in the prevention, alleviation and/or treatment of fibrosis.

A second aspect of the present invention relates to a composition for use in the prevention, alleviation and/or treatment of fibrosis, wherein said composition comprises a selective targeting agent as described herein and one or more physiologically acceptable carriers, excipients and/or diluents.

### BRIEF DESCRIPTION OF THE FIGURES

FIG. 1 shows that MFAP4 is increased in clinical/human fibrosis. MFAP4 immunostaining (brown) in A) control lung and lung with fibrosis, B) control kidney and renal fibrosis, and C) liver and liver fibrosis (from Molleken et al., 2009). D) Representative sample showing a subretinal fibrotic deposition (left panel) with Mfap4 mRNA expression in streaks of myofibroblasts shown by in situ hybridization (pink, right panel). Bl=blood, Fib=fibrotic deposition, myof=myofibroblasts. E) Representative sample showing −SMA (grey) positive myofibroblasts in fibrotic depositions in lung adenocarcinoma with Mfap4 mRNA expression shown by in situ hybridization (pink). Bars are in micrometers.

FIG. 2 shows that TGF-induces MFAP4 in transdifferentiating retinal pigment epithelial (RPE) cells and MFAP4 reversibly enhances growth factor-induced activation of these cells. TGF−-induced relative mRNA expression of A) MFAP4 or B) collagen in human RPE cells (ARPE19). MFAP4 tissue culture coating induced C) cellular adhesion (RFU=relative fluorescence units of fluorescently labelled cells sticking to surface) induced by MFAP4 was reversed by anti-MFAP4 treatment, D) PDGF-enhanced cellular migration through transwell filters coated with MFAP4 on filter side facing lower chamber, and E) TGF-induced collagen type 1 (Col1A1) mRNA synthesis was enhanced by MFAP4 after 24 hours and F) reversible by anti-MFAP4 treatment. C-F show data from 3-5 independent experiments. ANOVA was used to analyze the data. *p<0.05, **p<0.01, ****p<0.0001.

FIG. 3 shows that TGF-induces MFAP4 in transdifferentiating hepatic stellate cells and MFAP4 reversibly enhances growth factor-induced activation of these cells. A) TGF−-induced relative mRNA expression of MFAP4. MFAP4 tissue culture coating induced B) cellular adhesion and this attachment was reversed by anti-MFAP4 treatment. C) MFAP4 coating induced cellular collagen synthesis (tendency, preliminary data). D) PDGF-enhanced cellular migration through transwell filters coated with MFAP4 on filter side facing lower chamber, E) the migration was reduced with anti-MFAP4 treatment, and F) RGD-peptide treatment. ANOVA was used to analyze the data. *p<0.05, **p<0.01, ***, p<0.001, ****p<0.0001.

FIG. 4 shows that rat liver fibrosis is reduced by IV anti-MFAP4. A) Rat liver fibrosis was induced by two weeks phenobarbital in drinking water succeeded by 6 weeks carbontetrachloride (CCl₄) treatment by oral gavage. The rats received a total of three IV doses of anti-MFAP4 or vehicle treatment. B) The model reduced rat body weights, C) did not reduce the liver-to-body weight ratio, D) but significantly increased the spleen-to-body weight ratio (proxy for portal hypertension) and this effect was significantly reduced by anti-MFAP4 treatment. Immunodetection of MFAP4 was E) significantly induced in the model liver tissue and significantly reduced in this tissue, while insignificantly reduced in F) blood after anti-MFAP4 treatment. Relative mRNA expression of fibrotic and inflammatory markers G) collagen type 1 (Col1A1) mRNA, H)-smooth muscle actin (ACTA2), I) IL-1 (IL1), J) TGF-1 (TGF 1), K) TIMP-1 (TIMP1), and L) MFAP4 (MFAP4) mRNA was induced in rat liver tissue by the model and reduced with anti-MFAP4 treatment. Effects were significant for COL1a1 and TIMP1. ANOVA was used to analyze the data. *p<0.05, **p<0.01, ***, p<0.001, ****p<0.0001.

FIG. 5 shows that MFAP4 mediates Human primary HSCs (HHSteCs) adhesion and migration in vitro through RGD-integrin αvβ3 dependent interaction. (A-C) Cell adhesion performed using vibrant cell adhesion assay kit. HHSteCs (100,000 cells/well) were seeded and incubated for 1 hour onto A) 10 μg/ml of FN, HSA, rhMFAP4 or rhMFAP4 pretreated with MFAP4 blocking antibody, anti-MFAP4 (20 μg/ml); B) onto 10 μg/ml of rhMFAP4 in the presence of RGD or DGR containing peptides (100 μg/ml); C) onto 10 μg/ml of rhMFAP4 in competition with 10 μg/ml of integrin-blocking antibodies, anti-integrin αvβ3, αvβ5 or IC. (D-G) Migration assay was performed using transwell inserts. HHsteC (50,000 cells/inserts) were added to the upper chamber and allowed to migrate for 3h. Migrated cells were stained and counted under a light microscope (×20 magnification) in 4 random fields/inserts. The underside of the transwell inserts were coated with D) 10 μg/ml of HSA or rhMFAP4 in the presence or absence of PDGF in the lower chamber; E) with 10 μg/ml of HSA or rhMFAP4 in the presence of PDGF and 10 μg/ml anti-MFAP4 or IC in the lower chamber; (F-G) with 10 μg/ml HSA, FN or rhMFAP4 in the presence of PDGF in the lower chamber; cells were preincubated with (100 μg/ml) RGD or DGR containing peptides (F) or with 10 μg/ml of anti-integrin αvβ3 or IC. FN: Fibronectin, HSA: Human serum albumin, IC: isotype control, ns: not significant. Data are means+SEM of 3-4 independent experiments, each experiment is performed in triplicate. *p<0.05, **p<0.01, ***p<0,001, ****p<0,0001, calculated by one-way Anova.

FIG. 6 shows that MFAP4 is upregulated in HHSteCs upon activation with TGFβ1. MFAP4 enhances TGF 1 stimulated transdifferentiation of HHSteCs but no effect on HHSteCs proliferation. (A-D) 16 hours starved HHSteCs were cultured for 24, 48 or 72 h in the presence or absence of TGFβ1 (5 ng/ml) stimulation. A) MFAP4 concentration in culture supernatant determined by ELISA. (B-D) Relative mRNA expression of MFAP4, ACTA2 and Col1A1 in HHSteC+/−TGFβ1 stimulation. (E,F) 16 hours starved HHSteC were seeded on HSA or rhMFAP4 coated wells in the presence or absence of TGFβ1 stimulation and incubated for 72 h. Relative mRNA expression of ACTA2 (E) and Col1A1 (F). G) 16 hours starved HHSteC were seeded on HSA or rhMFAP4 coated wells and incubated for 4, 24, 48 or 72h, Cell's proliferation determined using WST-1 assay. Data are mean+SEM of 3 independent experiments. ns: not significant, **p<0.01, ***p<0,001, ****p<0,0001 calculated by one-way ANOVA or two-way ANOVA. FC: fold change.

FIG. 7 shows that MFAP4 increases in liver of CCl₄ induced liver fibrosis rat model in parallel with increase in SMA expression. MFAP4 concentration measured by ELISA in (A) liver and (B) serum of olive oil and CCl₄+vehicle treated rats (C) Relative mRNA expression for MFAP4 in liver of olive oil and CCl₄+vehicle treated rats. Data are mean+SEM, n=6 (olive oil group), n=10 (CCl₄+vehicle group). **p<0.01, ***p<0,001, calculated by t test.

FIG. 8 shows that neutralizing MFAP4 using an anti-MFAP4 antibody reduces liver fibrosis induced splenomegaly but not the levels of AST and ALT. A) Schematic representation of experimental model of liver fibrosis. B) Body weight of olive oil (n=6/group), CCl₄+vehicle and CCl₄+anti-MFAP4 (n=10/group), arrow represents the start of anti-MFAP4 or vehicle administration. Ratio of C) Liver and D) spleen to body weight in olive oil, CCl₄+vehicle and CCl₄+anti-MFAP4 treated rats. Enzymatic activity of E) ALT and F) AST detected in the serum of olive oil, CCl₄+vehicle or CCl₄+anti-MFAP4 treated rats using commercial ELISA kits. Data are mean+SEM, n=6 (olive oil group) n=10 (CCl₄+vehicle and CCl₄+anti-MFAP4). ns: not significant, *p<0.05, **p<0.01, calculated by one-way Anova.

FIG. 9 shows that targeting MFAP4 signaling using an anti-MFAP4 antibody reduces gene expression of CCl₄ induced liver profibrotic and proinflammatory markers. Relative mRNA expression level of A) Col1A1, B) ACTA2, C) TGFβ1, D) TIMP1, E) MMP2, F) IL1β, G) COL3A1 and H) MFAP4 in liver of olive oil, CCl₄+vehicle or CCl₄+anti-MFAP4 treated rats. RNA was extracted from liver homogenate of pieces from the left and right lobe. Tbp was used as housekeeping gene. Data are mean+SEM, n=6 (olive oil group), n=10 (CCl₄+vehicle and CCl₄+anti-MFAP4). Dotted line represents the mean of olive oil group. *p<0.05, **p<0.01, ***p<0,001, ****p<0,0001, calculated by t test or Mann-Whitney test.

FIG. 10 shows that anti-MFAP4 antibody protects from liver fibrosis progression in CCl₄ treated rats. A) Quantitative analysis of Sirius red positive areas (marker for collagen deposition). B) Concentration of the hydroxyproline content was measured in the liver homogenate. C) Quantification of −SMA positive areas (marker for myofibroblasts). D) Quantification of CD11b positive areas (marker for activated macrophages infiltrating the liver). E) Capillarization of liver sinusoids assessed by quantification of CD31 positive areas (marker for endothelial cells). Dotted line represents the mean of olive oil group. *p<0.05, **p<0.01, ***p<0,001, ****p<0,0001, calculated by one-way Anova, t-test or Mann-Whitney test.

FIG. 11 shows that anti-MFAP4 protects from liver fibrosis progression in non-alcoholic fatty liver disease/non-alcoholic steatohepatitis (NAFLD/NASH) in mice. Mouse liver fibrosis in NAFLD-model was induced by 7 weeks CCl₄ treatment+high fat diet, and the mice received 6 doses of IV anti-MFAP4 or vehicle for the last 3 weeks. Collagen staining of liver sections was obtained from vehicle or anti-MFAP4 treated animals, with quantification performed by image analyses.

The present invention will now be described in more detail in the following.

### DETAILED DESCRIPTION OF THE INVENTION

### Definitions

Prior to discussing the present invention in further details, the following terms and conventions will first be defined:

The term “fibrosis” also known as fibrotic scarring refers to a pathological wound healing in which connective tissue replaces normal parenchymal tissue leading to considerable tissue remodelling and formation of permanent scar tissue. Fibrosis involves fibroblasts laying down connective tissue including collagen and glycosaminoglycans in particular myofibroblastic cells. This process can lead to progressive irreversible fibrotic response if the tissue injury is severe or repetitive. Fibrosis may result as a consequence of repeated injuries, chronic inflammation and repair. Accordingly, fibrosis refers to the formation of excess fibrous connective tissue as a result of the excess deposition of extracellular matrix components like collagen. Collagen is present in fibrous connective tissue in a high content in the extracellular matrix. Thus, in fibrosis, there is a level of deposition of one or more extracellular matrix components, which is greater than the level of the absence of fibrosis.

In the present context, the term “treatment” refers to prophylactic treatment as well as therapeutic treatment.

In the present context, the term “alleviation” refers to making the disease i.e. the fibrosis less severe.

In the present context, the term “prevention” refers to the avoidance of the occurrence of fibrosis and may in some instances be considered as a prophylactic treatment.

The term “selective targeting agent” refers to the group of compounds, which target the MFAP4-protein and inhibit the integrin binding of MFAP4. This group relates to antibodies as well as antibody mimetic proteins and aptamers. The term “selective targeting agent” also includes fused compounds such as fusion proteins, where antibodies, aptamers or antibody mimetic proteins are fused to one or more other proteins.

The term “antibodies” as used herein is to be understood in the broad sense and is intended to cover also fragments or derivatives thereof. It is to be understood that in a fragment or derivative hereof the amino acid residues that interact with an antigen and confer its specificity and affinity for the antigen will be included i.e. that fragments and derivatives of antibodies are included as long as they exhibit the desired biological activity. Antibodies encompass immunoglobulin molecules and immunologically active portions of immunoglobulin (Ig) molecules. These include, but are not limited to, polyclonal antibodies, monoclonal antibodies, chimeric antibodies, a fully human antibody, a humanized antibody such as a humanized monoclonal antibody, an antibody wherein the heavy chain and light chain are connected by a flexible linker, single chain antibodies, Fc fragments, Fv fragments, scFv fragments, diabody, triabody, Fab fragments, Fab′ fragments, F(ab′)₂ fragments, conjugates having antibody CDR regions, nanobodies, fusion proteins, and a Fab expression library. The antibodies may be fully human antibodies, humanized antibodies or a chimeric antibody i.e. for example antibodies or fragments hereof that are specific to more than one source. The antibodies may also be part of fusion proteins, where the antibody is fused to another protein.

Preferably, the region binding to the antigen will be of human origin. In other embodiments, the region binding to the antigen can be derived from other animal species, in particular domestic animal and rodents such as rabbit, rat or hamster.

It is to be understood that each reference to “antibodies” or any like term herein includes intact antibodies as well as any fragments, alterations, derivatives, or variants thereof.

Antibody molecules relate to any of the classes IgG, IgM, IgA, IgE and IgD, which differ from one another by the nature of heavy chain present in the molecule. These include subclasses as well, such as IgG1, IgG2 and others. The light chain may be a kappa chain or a lambda chain. Reference herein to antibodies includes a reference to all classes, subclasses and types.

The antibodies may comprise moieties such as detectable labels or a substance having toxic or therapeutic activities. Alternatively, the antibodies may comprise point mutations in the constant region.

The term “monoclonal antibody” refers to an antibody obtained from a population of substantially homogeneous antibodies, wherein each monoclonal antibody will typically recognize a single epitope on the antigen. The term “monoclonal” is not limited to any particular method for making the antibody. For example, monoclonal antibodies of the invention may be made by the hybridoma method as described in (Köhler et al. 1975) or may be isolated from phage libraries using the techniques as described in WO 2019/086580.

The term “antibody mimetic proteins” as used herein relates to proteins, which are not antibodies as such but mimetic antibodies in their function by selectively targeting a specific target. As herein described, this relates to proteins, which specifically targets MFAP4 and inhibit the integrin binding of MFAP4. One example of an antibody mimetic protein is DARPins, which are genetically engineered antibody mimetic proteins that typically exhibit highly specific and high-affinity target protein binding. Other examples of such antibody mimetic proteins are affibody molecules, affilins, affimers, affitins, alphabodies, anticalins, avimers, fynomers, kunitz domain peptides, knottins, monobodies and nanoCLAMPs.

The term “aptamers” as used herein relates to oligonucleotides or peptide molecules that bind to a specific target molecule. Aptamers can be classified as DNA, RNA or XNA aptamer, which consist of strands of oligonucleotides and peptide aptamers, which are short variable peptide domains as known to persons skilled in the art. Aptamers are usually created by selecting them from a large random sequence pool based on their ability to bind to specific target molecules.

The terms “effective amount” and “therapeutically effective amount” when used in relation to a selective targeting agent refers to an amount of a selective targeting agent that is useful or necessary to support an observable change in the level of one or more biological activities of MFAP4, wherein said change may be either an increase or decrease in the level of MFAP4 activity.

In the context of the present invention, the term “sequence identity” or “homologue” indicates a quantitative measure of the degree of homology between two amino acid sequences or between two nucleic acid sequences. If the two sequences to be compared are not of equal length, they must be aligned to give the best possible fit, allowing the insertion of gaps or, alternatively, truncation at the ends of the polypeptide sequences or nucleotide sequences. The sequence identity can be calculated as

$$\frac{( {N}_{ref} - {N}_{dif} ) 100}{{N}_{ref}} ,$$

wherein Ndif is the total number of non-identical residues in the two sequences when aligned and wherein Nref is the number of residues in one of the sequences. Hence, the DNA sequence AGTCAGTC will have a sequence identity of 75% with the sequence AATCAATC (Ndif=2 and Nref=8). A gap is counted as non-identity of the specific residue(s), i.e. the DNA sequence AGTGTC will have a sequence identity of 75% with the DNA sequence AGTCAGTC (Ndif=2 and Nref=8).

With respect to all embodiments of the invention relating to amino acid sequences or nucleotide sequences, the percentage of sequence identity between one or more sequences may also be based on alignments using the clustalW software (http:/www.ebi.ac.uk/clustalW/index.html) with default settings. For nucleotide sequence alignments these settings are: Alignment=3Dfull, Gap Open 10.00, Gap Ext. 0.20, Gap separation Dist. 4, DNA weight matrix: identity (IUB). For amino acid sequence alignments, the settings are as follows: Alignment=3Dfull, Gap Open 10.00, Gap Ext. 0.20, Gap separation Dist. 4, Protein weight matrix: Gonnet.

Alternatively, nucleotide sequences may be analysed using programme DNASIS Max and the comparison of the sequences may be done at http://www.paralign.org/. This service is based on the two comparison algorithms called Smith-Waterman (SW) and ParAlign. The first algorithm was published by Smith and Waterman (1981) and is a well-established method that finds the optimal local alignment of two sequences. The other algorithm, ParAlign, is a heuristic method for sequence alignment; details on the method are published in Rognes (2001). Default settings for score matrix and Gap penalties as well as E-values were used.

In the present context, the terms “KD” or “K_{D} value” refer to the equilibrium dissociation constant between the selective targeting agent and its target for example between an antibody and its antigen. The K_{D} value relates to the concentration of selective targeting agent (the amount of selective targeting agent needed for a particular experiment) and so the lower the K_{D} value (lower concentration) and thus the higher the affinity of the selective targeting agent. In the present context, K_{D} is measured by Biacore T200.

In the context of the present invention, the definition “AA-yy referring to SEQ ID NO: X” is to be understood as AA=amino acid; −yy is the position of the amino acid in the SEQ ID NO: X. Thus, for example “Trp-33 referring to SEQ ID NO: 2” is to be understood as Trp at position 33 in SEQ ID NO: 2.

In the context of the present invention, the definition “at the most five amino acids” is to be understood as no more than five amino acids i.e. five amino acids, four amino acids, three amino acids, two amino acids or one amino acid.

In the context of the present invention, the definition “a sequence where at the most xx amino acids differ from the SEQ ID NO: X” is to be understood as the sequence being identical to the SEQ ID NO: X except for xx amino acids, which may be different i.e. a different amino acid than the one listed in the sequence. Thus, if at the most two amino acids differ from the SEQ ID NO: 9 this is to be understood as a sequence which differs from the SEQ ID NO: 9 by two, one or none amino acids. “X” is to be understood as any of the sequence listings SEQ ID NO: 1-15, such as any of the sequence listings SEQ ID NO: 1-14 as listed herein. Alternatively, “X” is to be understood as any of the sequence listings SEQ ID NO: 9, SEQ ID NO: 10, SEQ ID NO: 11, SEQ ID NO: 12, SEQ ID NO: 13, SEQ ID NO: 14. “xx” is to be understood as any of the numbers five, four, three, two or one.

In the context of the present invention, the term “composition” refers to a composition suitable for administration to a subject, which may comprise a pharmaceutically acceptable carrier or diluent. The active materials in the composition can be administered by any appropriate route, for example orally, parenterally, intravenously, intradermally, subcutaneously, systemically, intra-arterially, intramuscularly, intrathecally, intraocularly, intraconjuctivally, intravitreally or topically, in liquid or solid form. Such composition may also be known as a pharmaceutical composition.

If injectable, the composition may comprise the selective targeting agent in a sterile or isotonic medium.

Prevention, Alleviation and/or Treatment of Fibrosis

As mentioned above, this invention relates to selective targeting agents for preventing, alleviating and treating fibrosis. These selective targeting agents are directed against MFAP4 for binding hereto. These selective targeting agents are capable of targeting MFAP4 and hereby inhibits the action of MFAP4 integrin binding. Fibrosis can hereby be prevented as well as reversed as demonstrated in the examples by a direct interaction of the selective targeting agents with the components of the extracellular matrix abundantly expressed during fibrosis and i.e. a clear marker of fibrosis such as collagen. Furthermore, the results demonstrate that treatment with the selective targeting agents also is able to reverse the phenotype of the cells responsible for the fibrotic process and hence, to directly affect the fibrotic process. Thus in contrast to available drugs, fibrosis may be treated by actually reversing the fibrotic process. Accordingly, in a first aspect, the invention relates to a selective targeting agent, which is capable of binding MFAP4 and inhibiting MFAP4-integrin interaction for use in the prevention, alleviation and/or treatment of fibrosis.

In one embodiment, the selective targeting agent is selected from the group consisting of an antibody, an antibody mimetic protein and an aptamer.

In a further embodiment, the selective targeting agent is an epitope-binding protein such as an antibody or an antibody mimetic protein. The term “epitope-binding protein” refers to the group of proteins, which target MFAP4 and inhibit the integrin binding of MFAP4 by binding to an epitope in MFAP4. This group relates to antibodies as well as antibody mimetic proteins. The term “epitope-binding protein” also includes fusion proteins, where antibodies or antibody mimetic proteins are fused to one or more other proteins.

Fibrosis may be introduced in different tissues such as liver, lungs, kidney, heart, blood vessels, eye, skin, pancreas, intestine, brain, and bone marrow caused e.g. by injuries to this particular tissue. Fibrosis may also occur in several organs at the same time.

As an example, lung fibrosis or pulmonary fibrosis may occur as a result of long-standing infections such as tuberculosis or pneumonia. It could also be caused by exposure to occupational hazards such as coal dust or the genetic condition cystic fibrosis. Liver fibrosis and cirrhosis is scar tissue that replace normal liver tissue. Liver cirrhosis is defined as an advanced stage of liver fibrosis with distortion of the hepatic vasculature and architecture. Liver fibrosis and cirrhosis can be a consequence of different causes, such as obesity and the metabolic syndrome leading to fatty liver disease (FLD), non-alcoholic fatty liver disease (NAFLD) or metabolic associated fatty liver disease (MAFLD) which also comprise non-alcoholic steatohepatitis (NASH); high alcohol consumption leading to alcoholic fatty liver disease (AFLD) or alcoholic steatohepatitis (ASH); hepatitis B or C infection; autoimmune diseases; cholestatic diseases; and iron or copper overload. Also, the heart may suffer from fibrosis, where areas of the heart have become damaged due to myocardial infarction, pressure overload or other cause.

Thus, the selective targeting agent may be used for treating, alleviating and/or preventing fibrosis in different tissues. In one embodiment, fibrosis is liver fibrosis or chronic liver diseases, lung fibrosis, eye fibrosis, kidney fibrosis, heart fibrosis, intestinal fibrosis and/or fibrosis in response to surgery or injury.

In a further embodiment, fibrosis is liver fibrosis or chronic liver diseases. In an even further embodiment, said chronic liver diseases are cirrhosis, liver failure and portal hypertension. Thus, by liver fibrosis is to be understood that the fibrosis is present in the liver.

In a further embodiment, fibrosis is lung fibrosis. Hereby is to be understood that the fibrosis is present in the lung.

In a further embodiment, fibrosis is eye fibrosis. Hereby is to be understood that the fibrosis is present in the eye. Eye fibrosis may be retinal fibrosis including subretinal, epiretinal and preretinal fibrosis, conjunctival fibrosis, corneal fibrosis, fibrosis of trabecular meshwork and in the lens, and fibrosis in response to surgery or injury in any location of the eye.

In a further embodiment, fibrosis is kidney fibrosis. Hereby is to be understood the fibrosis is present in the kidney.

In a further embodiment, fibrosis is heart fibrosis. Hereby is to be understood that the fibrosis is present in the heart. Heart fibrosis may be associated with dysfunction of the musculature or electrical properties of the heart or thickening of the walls of the valves of the heart. In some embodiments, heart fibrosis is of the atrium and/or ventricles of the heart. Treatment, alleviation or prevention of heart fibrosis may reduce risk of atrial fibrillation, ventricular fibrillation or myocardial infarction.

In a further embodiment, fibrosis is intestinal fibrosis. Hereby is to be understood that the fibrosis is present in the intestinal system. In one embodiment, fibrosis in the intestinal system develops in connection with stenotic anastomosis. In a further embodiment, the fibrosis in the intestinal system could be a consequence of surgery or injury.

In a further embodiment, fibrosis is a response to surgery. Hereby is to be understood that the fibrosis arises as a consequence of surgery. In one embodiment, fibrosis develops in connection with stenotic anastomosis.

In a further embodiment, fibrosis in a response to injury. Hereby is to be understood that the fibrosis arises as a consequence of injury.

Several diseases involves fibrosis and may be caused by fibrosis such as but not limited to: arthrofibrosis; Dupuytren's contracture; mediastinal fibrosis; retroperitoneal fibrosis; myelofibrosis; Peyronie's disease; adhesive capsulitis; kidney disease (e.g. renal fibrosis, nephritic syndrome, diabetic nephropathy, chronic glomerulonephritis, nephritis associated with systemic lupus); progressive systemic sclerosis (PSS); chronic graft versus host disease; diseases of the eye (e.g. epiretinal fibrosis, retinal fibrosis, subretinal fibrosis, conjunctival fibrosis, subconjunctival fibrosis); arthritis; fibrotic pre-neoplastic and fibrotic neoplastic disease; respiratory conditions (e.g. pulmonary fibrosis, cystic fibrosis, idiopathic pulmonary fibrosis, progressive massive fibrosis, and asthma); liver diseases (e.g. chronic liver disease, primary biliary cirrhosis (PBC), schistosomal liver disease, liver cirrhosis); cardiovascular conditions (e.g. hypertrophic cardiomyopathy, dilated cardiomyopathy (DCM), fibrosis of the atrium, atrial fibrillation, fibrosis of the ventricle, ventricular fibrillation, myocardial fibrosis, myocarditis, endomyocardial fibrosis, myocardial infarction, fibrotic vascular disease, tubulointerstitial and glomerular fibrosis, atherosclerosis,); neurological conditions (e.g. gliosis and Alzheimer's disease); muscular dystrophy (e.g. Duchenne muscular dystrophy (DMD) or Becker's muscular dystrophy (BMD)); gastrointestinal conditions (e.g. Chron's disease, microscopic colitis and primary sclerosing cholangitis (PSC)); skin conditions (e.g. scleroderma, nephrogenic systemic fibrosis and cutis keloid); and fibrosis induced by chemical or environmental insult (e.g., cancer chemotherapy, pesticides, radiation/cancer radiotherapy).

### Effect on Myofibroblasts and Fibrotic Deposition Such as Collagen

The selective targeting agents targeting MFAP4 are able to reverse the myofibroblastic activation and hereby prevent the formation of fibrotic deposition such as collagenous tissue formation as demonstrated by the examples as disclosed herein. Hence, in on embodiment, the present invention relates to a selective targeting agent, which is capable of binding MFAP4 and inhibiting MFAP4-integrin interaction for use in de-differentiating myofibroblasts.

The treatment with selective targeting agents is even capable of reversing the amount of collagen expressed. Thus, in on embodiment, the present invention relates to a selective targeting agent, which is capable of binding MFAP4 and inhibiting MFAP4-integrin interaction for use in reducing and/or reversing synthesis of collagen. In a further embodiment, the collagen is collagen type I or collagen type III. In an even further embodiment, the collagen is collagen type I.

By reducing is meant that less fibrotic deposition such as collagen is synthesized than what is normally produced by the healthy tissue, while reversing means that increased amounts of collagen in the tissue e.g. due to fibrosis is reduced to a level of collagen observed in the healthy tissue prior to this development of the specific pathology. Thus, tissues characterised by an increased amount of extracellular matrix material such as i.e. fibrosis can be prevented or treated.

### Selective Targeting Agent

Selective targeting agents targeting MFAP4 may have different sequences and different binding properties but they are able to bind to MFAP4 and capable of preventing MFAP4 from binding to integrins.

The selective targeting agents of the invention bind selectively to MFAP4 that is they bind preferentially to MFAP4. The selective targeting agents may bind selectively to human MFAP4, but also bind detectably to non-human MFAP4, such as murine MFAP4. Alternatively, the selective targeting agents may bind exclusively to human MFAP4, with no detectable binding to non-human MFAP4.

It is to be understood that the selective targeting agent used according to this invention may be either one type of selective targeting agent or a mixture of selective targeting agents capable of exerting a similar effect.

In one embodiment, the selective targeting agent is selected from the group consisting of antibodies, antibody mimetic protein and aptamers.

It may also be advantageous to couple different moieties to the selective targeting agent according to the invention. Thus, in an embodiment, the selective targeting agent is coupled to a detectable label or a substance having toxic or therapeutic activity.

For the selective targeting agents such as antibodies to perform as medicaments against fibrosis and/or synthesis of collagen, they have to be sufficiently soluble and not form aggregates, which may precipitate. Thus, in yet an embodiment, the selective targeting agent is primarily in a monomeric form (measured in PBS, pH 7.4). “Primarily” is to be understood as at least 90% of the antibodies are in monomeric form, such as at least 95%.

In one embodiment, the selective targeting agent does not bind (directly) to the RGD-integrin interaction sequence in rhMFAP4, but still block MFAP4-mediated activity suggested by steric hindrance of integrin ligation.

In a further embodiment, the selective targeting agent has a K_{D} value to rhMFAP4 below 1*10⁻⁷, such as below 1*10⁻⁸, or such as below 1*10⁻⁹ M, or such as in the range 1*10⁻⁷ to 1*10⁻¹² M, such as in the range 1*10⁻⁷ to 1*10⁻¹⁰ M as measured by Biacore T200.

### Antibodies

In one embodiment, the selective targeting agent is an antibody. Antibody is to be understood in the broad sense i.e. including not only the full antibody but also derivatives or fragments. These antibodies include humanized antibodies. An antibody for targeting MFAP4 is also mentioned as anti-MFAP4 herein. These antibodies may have different sequences and different binding properties but they are able to bind to MFAP4 and capable of preventing MFAP4 from binding to integrins.

The antibodies may be produced in different forms. In one embodiment, the anti-MFAP4 is an antibody or a fragment hereof selected from the group consisting of polyclonal antibodies, monoclonal antibodies, chimeric antibodies, a fully human antibody, a humanized antibody such as a humanized monoclonal antibody, an antibody wherein the heavy chain and light chain are connected by a flexible linker, single chain antibodies, Fc fragments, Fv fragments, scFv fragments, diabody, triabody, Fab fragments, Fab′ fragments, F(ab′)₂ fragments, conjugates having antibody CDR regions, nanobodies, fusion proteins, and a Fab expression library. In one embodiment, the antibody is a F(ab′)₂ molecule. In another embodiment, the antibody is selected from the group consisting of a monoclonal antibody, a Fab fragment and a humanized monoclonal antibody. In yet another embodiment, the antibody is humanized and/or monoclonal, preferably a humanized monoclonal antibody.

Examples of antibodies are
mAS0326 having a light chain variable region according to SEQ ID NO: 1 and a heavy chain variable region according to SEQ ID NO: 2
hAS0326 having a light chain variable region according to SEQ ID NO: 3 and a heavy chain variable region according to SEQ ID NO: 4
HG Hyb 7-14 having a light chain variable region according to SEQ ID NO: 5 and a heavy chain variable region according to SEQ ID NO: 6
HG Hyb 7-5 having a light chain variable region according to SEQ ID NO: 7 and a heavy chain variable region according to SEQ ID NO: 8

In one embodiment, HG Hyb 7-5, HG Hyb 7-14, mAS0326 and hAS0326 are used in the first aspect according to this invention.

In one embodiment, the antibody comprises
a light chain variable region comprising the amino acid sequence of SEQ ID NO: 1, 3, 5 or 7, or sequences having at least 80% sequence identity, such as at least at least 90% sequence identity, or such as at least 95% sequence identity to SEQ ID NO: 1, 3, 5, or 7; and
a heavy chain variable region comprising the amino acid sequence of SEQ ID NO: 2, 4, 6 or 8, or sequences having at least 80% sequence identity, such as at least 90% sequence identity, or such as at least 95% sequence identity to SEQ ID NO: 2, 4, 6, or 8.

In one embodiment, the antibody comprises
a light chain variable region comprising the amino acid sequence of SEQ ID NO: 1, 3, 5, or 7, or sequences having at least 80% sequence identity, such as at least 90% sequence identity, or such as at least 95% sequence identity to SEQ ID NO: 1, 3, 5, or 7; and
a heavy chain variable region comprising the amino acid sequence of SEQ ID NO: 2, 4, 6, or 8, or sequences having at least 80% sequence identity, such as at least 90% sequence identity, or such as at least 95% sequence identity to SEQ ID NO: 2, 4, 6, or 8.

In a further embodiment, the antibody comprises
a light chain variable region comprising the amino acid sequence of SEQ ID NO: 5, or sequences having at least 80% sequence identity, such as at least at least 90% sequence identity, or such as at least 95% sequence identity to SEQ ID NO: 5; and
a heavy chain variable region comprising the amino acid sequence of SEQ ID NO: 6, or sequences having at least 80% sequence identity, such as at least 90% sequence identity, or such as at least 95% sequence identity to SEQ ID NO: 6.

In a further embodiment, the antibody comprises
a light chain variable region comprising the amino acid sequence of SEQ ID NO: 7, or sequences having at least 80% sequence identity, such as at least at least 90% sequence identity, or such as at least 95% sequence identity to SEQ ID NO: 7; and
a heavy chain variable region comprising the amino acid sequence of SEQ ID NO: 8, or sequences having at least 80% sequence identity, such as at least 90% sequence identity, or such as at least 95% sequence identity to SEQ ID NO: 8.

In a further embodiment, the antibody comprises
a light chain variable region comprising the amino acid sequence of SEQ ID NO: 1, or sequences having at least 80% sequence identity, such as at least at least 90% sequence identity, or such as at least 95% sequence identity to SEQ ID NO: 1; and
a heavy chain variable region comprising the amino acid sequence of SEQ ID NO: 2, or sequences having at least 80% sequence identity, such as at least 90% sequence identity, or such as at least 95% sequence identity to SEQ ID NO: 2.

In a further embodiment, the antibody comprises
a light chain variable region comprising the amino acid sequence of SEQ ID NO: 3, or sequences having at least 80% sequence identity, such as at least at least 90% sequence identity, or such as at least 95% sequence identity to SEQ ID NO: 3; and
a heavy chain variable region comprising the amino acid sequence of SEQ ID NO: 4, or sequences having at least 80% sequence identity, such as at least 90% sequence identity, or such as at least 95% sequence identity to SEQ ID NO: 4.

In an even further embodiment, the antibody comprises
a light chain variable region comprising
   a CDR 1 region according to SEQ ID NO: 9 or according to a sequence where at the most five amino acids differ, such as at the most four amino acids differ, like at the most three amino acids differ, such as at the most two amino acids differ, like at the most one amino acid differ from SEQ ID NO: 9 with the proviso that the amino acid at position 9 is a Tyr;
   a CDR 2 region according to SEQ ID NO: 10 or according to a sequence where at the most five amino acids differ, such as at the most four amino acids differ, like at the most three amino acids differ, such as at the most two amino acids differ, like at the most one amino acid differ from SEQ ID NO: 10; and
   a CDR 3 region according to SEQ ID NO: 11 or according to a sequence where at the most five amino acids differ, such as at the most four amino acids differ, like at the most three amino acids differ, such as at the most two amino acids differ, like at the most one amino acid differ from SEQ ID NO: 11 with the proviso that the amino acid at position 6 is a Tyr; and
a heavy chain variable region comprising
   a CDR 1 region according SEQ ID NO: 12 or according to a sequence where at the most five amino acids differ, such as at the most four amino acids differ, like at the most three amino acids differ, such as at the most two amino acids differ, like at the most one amino acid differ from SEQ ID NO: 12 with the proviso that the amino acid at position 3 is a Trp and optionally with the proviso that the amino acid at position 4 is a Met;
   a CDR 2 region according to SEQ ID NO: 13 or according to a sequence where at the most five amino acids differ, such as at the most four amino acids differ, like at the most three amino acids differ, such as at the most two amino acids differ, like at the most one amino acid differ from SEQ ID NO: 13, optionally with the proviso that the amino acid at position 4 is a Pro; and
   a CDR 3 region according to SEQ ID NO: 14 or according to a sequence where at the most five amino acids differ, such as at the most four amino acids differ, like at the most three amino acids differ, such as at the most two amino acids differ, like at the most one amino acid differ from SEQ ID NO: 14 with the proviso that the amino acid at position 1 is a Glu and the amino acid at position 9 is a Trp.

In an even further embodiment, the antibody comprises
a light chain variable region comprising a CDR 1 region according to SEQ ID NO: 9, a CDR 2 region according to SEQ ID NO: 10 and a CDR 3 region according to SEQ ID NO: 11; and
a heavy chain variable region comprising a CDR 1 region according to SEQ ID NO: 12, a CDR 2 region according to SEQ ID NO: 13 and a CDR 3 region according to SEQ ID NO: 14.

In a still further embodiment, the antibody comprises
a light chain variable region comprising the amino acid sequence of SEQ ID NO: 1 or 3, or sequences having at least 80% sequence identity, such as at least at least 90% sequence identity, like at least 95% sequence identity, such as 98% sequence identity, or like 99% sequence identity to SEQ ID NO: 1 or 3 with the proviso that the amino acid at position 32 is a Tyr and the amino acid at position 94 is a Tyr;
a heavy chain variable region comprising the amino acid sequence of SEQ ID NO: 2 or 4, or sequences having at least 80% sequence identity, such as at least 90% sequence identity, like at least 95% sequence identity, such as at least 98% sequence identity or like 99% sequence identity to SEQ ID NO: 2 or 4 with the proviso that the amino acid at position 33 is a Trp, the amino acid at position 99 is a Glu and the amino acid at position 107 is a Trp and optionally, with the proviso that the amino acid at position 34 is a Met the amino acid at position 53 is a Pro.

It is to be understood that the antibodies may be variants and/or fragments of the antibodies as described above.

The antibodies may be produced by standard methods as commonly known to the skilled person in the art. As an example, the antibodies may be expressed by one or more vectors such as plasmid(s). It is to be understood that e.g. the light chain or heavy chain may be expressed from two different vectors. In an embodiment, the vector is a plasmid. The vectors may be expressing the antibody in a cell such as CHO cells.

### Antibody Mimetic Protein

In one embodiment, the selective targeting agent is an antibody mimetic protein. In a further embodiment, the antibody mimetic protein selected from the group consisting of affibody molecules, affilins, affimers, affitins, alphabodies, anticalins, avimers, DARPins, fynomers, kunitz domain peptides, knottins, monobodies and nanoCLAMPs.

In a further embodiment, the selective targeting agent is DARPins. DARPins are genetically engineered antibody mimetic proteins that typically exhibit highly specific and high-affinity target protein binding.

The antibody mimetic protein may be produced by standard methods as commonly known to the skilled person in the art.

### Aptamers

In one embodiment, the selective targeting agent is an aptamer. In a further embodiment, the aptamer is selected from the group consisting of a DNA aptamer, a RNA aptamer, an XNA aptamer and a peptide aptamer.

The aptamer may be produced by standard methods as commonly known to the skilled person in the art.

### Composition

The selective targeting agent may be in a composition (such as a pharmaceutical composition). Thus, in a second aspect, the invention relates to a composition for use in the prevention, alleviation and/or treatment of fibrosis, wherein said composition comprises a selective targeting agent as described herein and one or more physiologically acceptable carriers, excipients and/or diluents.

In an embodiment, said composition comprises one or more stabilizing agents and/or one or more buffering agents. In yet an embodiment, the stabilizing agent is a surfactant.

### Administration

For the selective targeting agent and/or the composition to be able to prevent, alleviate and/or treat fibrosis as well as to reduce and/or reverse the collagen production, an effective amount of the selective targeting agent and/or composition is to be provided to the subject to be treated. The effective amount depends on the size of the subject to be treated as well as the method of administration and the degree of criticality of the disease. The effective amount may be adjusted by the skilled person.

The subject to be treated is to be understood as any subject capable of developing fibrosis. In one embodiment, the subject is a mammal. In a preferred embodiment, the mammal is a human.

The selective targeting agent and/composition may be administered to the subject to be treated in different ways commonly known in the art. Thus, in one embodiment, the selective targeting agent and/or the composition is administered orally, parenterally, intravenously, intradermally, subcutaneously, systemically, intra-arterially, intramuscularly, intrathecally, intraocularly, intraconjuctivally, intravitreally, intranasally, by inhalation, topically or by direct or catheter guided local organ administration. In a further embodiment, the selective targeting agent and/or the composition is administered in liquid or solid form.

In a further embodiment, the selective targeting agent and/or the composition is administered by intravenous, intravitreal or intraperitoneal injection.

In a further embodiment, the selective targeting agent and/or composition is administered by injection or inhalation into the fibrotic tissue. Hereby, the selective targeting agent and/or composition is delivered directly to the affected area and can be thus, act locally on the specific tissue affected by increased collagen synthesis and/or fibrosis. This may also lead to the use of less selective targeting agent and/or composition to achieve the similar effect.

The effect of the selective targeting agent and/or composition may decrease over time, thus depending on the condition to be treated; several times of administration may be needed. Thus, in one embodiment, the selective targeting agent and/or the composition is administered at least once, such as at least twice, such as at least three times, such as at least four times, such as at least 5 times, such as at least 6 times, such as at least 7 times, such as at least 8 times, such as at least 9 times, such as at least 10 times. In a further embodiment, the selective targeting agent and/or the composition is administered three times or four times. In a further embodiment, the selective targeting agent and/or the composition is administered lifelong. This is particular relevant for cases of chronic diseases that the selective targeting agent and/or the composition is administered the entire lifetime of the subject.

In a further embodiment, the selective targeting agent and/or the composition is administered by injection at regular intervals. In an even further embodiment, the selective targeting agent and/or the composition is administered at the most once a week, such as at the most once every 2 weeks, such as at the most once every 4 weeks, such as at the most once every 6 weeks, such as at the most once every 8 weeks, such as at the most once every 10 weeks, such as at the most once every 12 weeks, such as at the most once every 14 weeks, such as at the most once every 16 weeks, such as at the most every once 20 weeks, such as at the most every once every 6 months, such as at the most once every year. In a further embodiment, the selective targeting agent and/or the composition is administered once a week.

In a still further embodiment, the selective targeting agent and/or the composition is administered by injection once a week for at least three weeks, such as three weeks or four weeks.

### Method of Prevention or Treatment of Fibrosis

In yet another aspect, the invention relates to a method of prevention or treatment of fibrosis, said method comprising administering the selective targeting agent or the composition according to the invention to a subject (in need thereof). In one embodiment, said subject is a mammal. In a preferred embodiment, said mammal is a human.

In a further embodiment, the subject (in need thereof) is diagnosed with one of the diseases involving fibrosis as mentioned herein.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Materials and Methods

### Human Tissues

Formalin-fixed and paraffin-embedded human eye samples with age-related macular degeneration or diabetic retinopathy (total n=6) were retrieved from the tissue bank of the Eye Pathology Institute, University of Copenhagen. Patients had enucleation carried out because of pain. Some eyes showed histological presence of serous subretinal neovascular membrane and/or a subretinal haemorrhage and/or a periretinal fibrous scar. Human lung adenocarcinoma tissues were obtained from the diagnostic Biobank at the Department of Pathology, Odense University Hospital (Odense, Denmark).

### Anti-MFAP4 Used

High-affinity IgG1 Mabs, anti-MFAP4, are produced using mouse immunization of MFAP4-deficient mice and standard hybridoma technique (Wulf-Johansson et al., 2013).

hrMFAP4 used e.g. for immunization was produced as previously described (Sokmose et al., 2013)

Monoclonal antibodies with affinity for hrMFAP4 were produced using standard hybridoma technique and MFAP4 deficient mice.

This procedure resulted in a series of monoclonal antibodies including:
HG Hyb 7-5 murine monoclonal antibody raised against human recombinant MFAP4 (hrMFAP4) (described e.g. in WO 2014/114298)
HG Hyb 7-14 murine monoclonal antibody raised against human recombinant MFAP4 (hrMFAP4) (described e.g. in WO 2014/114298)
HG Hyb 7-18 murine monoclonal antibody raised against human recombinant MFAP4 (hrMFAP4) (described e.g. in WO 2014/114298)
mAS0326 murine monoclonal antibody raised against human recombinant MFAP4 (hrMFAP4) and chosen antibody for humanization
hAS0326 humanized monoclonal antibody (described e.g. in WO 2019/086580)
hAS0326 Fc-neutralized humanized monoclonal antibody, which is similar to hAS0326, except for point mutations in the Fc-domain of the heavy chain. These point mutations were introduced using standard techniques as known to the persons skilled in the art.

All antibodies were amino acid sequenced using standard techniques.

### Immunohistochemistry

Anti-MFAP4 (HG-HYB 7-14) was labeled with FITC (isomer 1; Sigma-Aldrich, Saint Louis, MO) according to manufacturer's instructions. Four-μm-thick sections were cut from NBF-fixed paraffin-embedded tissue blocks. Sections were mounted on FLEX IHC Slides (Dako; Glostrup, Denmark), dried at 60° C., dewaxed, and rehydrated through a graded ethanol series, and subsequently washed in 0.05 M Tris-buffered saline (TBS; Fagron Nordic A/S; Copenhagen, Denmark). Epitope retrieval was performed by protease treatment (0.05% protease type XIV; Sigma-Aldrich, St Louis, MO) for 15 min. The FITC-labeled antibodies were diluted to 2.5 μg/ml in Antibody Diluent (Agilent Technologies; Glostrup, Denmark). Incubation with the antibodies was done for 60 min at room temperature followed by incubation with anti-FITC-horseradish peroxidase (HRP) (Dako, Glostrup, Denmark), diluted 1:30, and incubated for 20 min. Immunostaining was followed by brief nuclear counterstaining in Mayer's hematoxylin (Fagron Nordic A/S; Copenhagen, Denmark). Finally, slides were washed, dehydrated, and coverslipped using a Tissue-Tek Film coverslipper (Sakura Finetek; Alphen aan den Rijn, The Netherlands).

Positive and negative controls for immunostaining of MFAP4 using antibody HG-HYB 7-14 was previously shown using Mfap4^{+/+} (wild-type) and Mfap4^{-/-} (gene-deficient mice) (Schlosser et al., 2016).

### RNA In Situ Hybridization (ISH)

RNA ISH was conducted on human tissues essentially as described in (Lassen et al., 2017) using a modified version of the RNAScope 2.5 high-definition procedure (Cat. No. 322300, Advanced Cell Diagnostics [ACD] bioscience, Newark, CA).

4-μm-sections of the tissues were in situ hybridized with 20 probe pairs (Cat. No. 571221, ACD) targeting nucleotide 109-1150 of human MFAP4 mRNA (GenBank accession number: NM_002404.2) followed by branched-DNA signal amplification and tyramide-enhancement visualized with Liquid Permanent Red (Cat. No. K0640; Agilent). Subsequently, some sections were immunostained with mouse anti-αSMA antibodies (Cat. No. M0851; Agilent) detected with HRP anti-mouse BrightVision (ImmunoLogic) and visualized with Deep Space Black (BioCare).

### ARPE-19 Studies

### Cell Culture:

Human retinal pigment epithelial cell line ARPE-19 (ATCC) was cultured in DMEM/F-12 medium supplemented with 10% FBS, 2 mM L-glutamine, 50 U/ml penicillin and 50 μg/ml streptomycin. Cells were subcultured every 3-4 days.

Cell Stimulation and qPCR

ARPE-19 cells were seeded (250.000 cells/well) in a 12-well plate, allowed to adhere for 8 h and then serum-starved in overnight. The cells were then stimulated with increasing concentrations of rTGF-β2 for 24 and 48 h. Alternatively, cells were seeded onto MFAP4-coated and albumin-blocked wells, serum-starved and incubated with rTGF-β2 for 24 h. In some experiments, the wells were incubated with 10 g/ml hAS0326 (anti-MFAP4) antibody before seeding the cells. Total RNA was extracted using Trizol reagent according to the manufacturer's instruction. Reverse transcription was performed using 1 μg isolated RNA and M-MLV Reverse Transcriptase according to the manufacturer's instruction. Real-time PCR was performed using the Tagman Universal PCR Master Mix and Taqman Gene Expression Assays (ThermoFischerScientific) using the following primers: hGAPDH, Assay ID: Hs99999905 (Cat. No. 4331182; ThermoFisherScientific); hMFAP4, Assay ID: Hs00412974 (Cat. No. 4331182; ThermoFisherScientific); hCOL1A1, Assay ID: Hs00164004 (Cat. No. 4331182; ThermoFisherScientific).

### Adhesion Assay

Cell adhesion was assessed using the Vybrant™ Cell Adhesion Assay Kit (Molecular Probes, Invitrogen) according to the manufacturer's instructions. Briefly, a black 96-well plate (Nunc) was coated with MFAP4, fibronectin (Sigma-Aldrich) or albumin (Sigma-Aldrich) at 4° C. overnight, washed with PBS, blocked with 10 mg/ml sterile albumin for 1 hour at room temperature (RT) and washed again. Calcein-labeled cell suspensions were loaded on to the pre-coated plate at 1-2·10⁵ cells/well. In some experiments, the plate was incubated with 10 μg/ml hAS0326 (anti-MFAP4) or isotype control antibody (human IgG1, BioXCell) for 1 hour at room temperature. After 1 hour of incubation at 37° C. the plate was washed four times with warm medium and filled with 200 μl PBS. The fluorescence was measured at 485/535 nm using a VICTOR™ 3 Multilabel plate Reader (Perkin Elmer). All samples were normalized to blank wells containing PBS only (Sigma-Aldrich).

### Migration Assay

Lower side of the Transwell inserts with 8.0 μm pores (Corning) was coated with 10 μg/cm² MFAP4 or albumin overnight at 4° C. and washed with PBS. ARPE-19 cells were collected, resuspended in DMEM/F-12 medium containing 0.5% FBS and seeded in the upper chamber of the inserts (50.000 cells/insert). Lower chamber contained the medium with 0.5% FBS±ng/ml PDGF. The cells were allowed to migrate for 24 h, after which the upper side of the filters were washed with PBS and swiped with a cotton swab to remove any non-migrated cells. The lower side of the filters were stained with Hemacolor (Sigma-Aldrich) and divided into four fields. The cells in each field were counted in a blinded manner by two independent observers.

In some experiments, full-length hAS0326 (Anti-MFAP4, 10 μg/ml) antibody were added to the lower chamber. Alternatively, anti-integrin α_{V}β₃ (Millipore) anti-integrin α_{V}β₅ (Millipore) or isotype control antibody (mouse IgG1, Thermofisher) were added to the upper chamber when seeding the cells.

### Hepatic Stellate Cell Studies

### Cell Culture

Human primary HSCs (HHSteCs, ScienCell Research Laboratories/3H biomedical) were used for the in vitro work. Cells were cultured in stellate cell medium+2% fetal bovine serum (FBS)+1% stellate cells growth supplements (SteCGs, ScienCell Research Laboratories/3H biomedical). HHSteCs were passaged when 80-90% confluent and were used between passage 3 and 10 for the experiments.

Cell Stimulation and qPCR

Prior to MFAP4 or TGF-β1 stimulation, cells were starved in Stellate cell medium+0% FBS and 0% SteCGs for 16 hrs. For MFAP4 stimulation, 6 well plates were coated with 10 μg/ml MFAP4 or 10 μg/ml human serum albumin (HSA) (Sigma-Aldrich) in PBS overnight at 4° C. The next day, wells were washed and blocked for 1 h with 10 mg/ml HSA. HHSteC were seeded at a density of 200,000 cells/well and incubated for 48 hours. For TGF-β1 stimulation, HHSteC were seeded at a density of 200,000 cells/well in 6 well plate in growth medium. The following day cells were washed twice with PBS and medium was changed to 0% FBS in SteCM without growth supplements for 16 hours, then cells were stimulated with ng/ml recombinant human TGF-β1 for 72 h. Real-time PCR was performed as described above.

### Adhesion Assay

Black 96-well Maxisorb plates were coated at 4° C. overnight with 10 μg/ml recombinant human MFAP4 (rhMFAP4), 10 μg/ml human bovine serum (HSA) (Sigma-Aldrich) and 10 μg/ml fibronectin (FN) (Sigma-Aldrich) in PBS. The next day, wells were washed and blocked for 1 h with 10 mg/ml HSA. For blocking of MFAP4, immobilized rhMFAP4 was incubated with 20 μg/ml anti-MFAP4 antibody hAS0326 for 1 h at room temperature before seeding cells. For experiments assessing the involvement of RGD site and integrins, cells were incubated for 30 min at room temperature with synthetic 100 μg/ml RGD (Sigma-Aldrich) or 100 μg/ml DGR peptides (a modified form of RGD used as control) (Sigma-Aldrich) or 10 μg/ml of anti-integrin αvβ3 (Sigma-Aldrich), αvβ5 (Sigma-Aldrich), αvβ6 (Sigma-Aldrich) or isotype control (IC) (anti-ovalbumin, HYB099-01; Statens Serum Institut, Copenhagen, Denmark).

Vybrant cell adhesion assay Kit (Molecular Probes, Invitrogen) was used for the quantification of cell adhesion. Cells were detached using trypsin and resuspended in serum free medium containing 5 μM Calcein AM dye for 5 million cells/ml, and incubated for 30 min at 37° C. After incubation, cells were washed twice with PBS and seeded at a density of 100,000 cell/well and incubated for 1 h at 37° C. Then cells were washed and 200 μl PBS was added to each well and the fluorescence was measured using the Gudlaug-Fluorescein (485 nm/535 nm, 1.0s) protocol on the victor plate reader.

### Migration Assay

The underside of the membrane of transwell inserts with 8-μm pore size were coated with 10 μg/ml rhMFAP4 or HSA overnight at 4° C. HHSteCs (100,000 cell/insert) were seeded in the upper chamber in serum-free medium and they were allowed to migrate for 3h to the lower chamber containing serum free medium+/−100 ng/ml Platelet Derived Growth Factor (PDGF) (R&D systems). For MFAP4 blocking experiments, 10 μg/ml of hAS0326 or 10 μg/ml isotype control (IC) were added to the lower chamber. For experiments testing the role of RGD site and integrins, cells were pre-incubated with synthetic 100 μg/ml RGD or DGR peptides or 10 μg/ml anti-integrin αvβ3 or isotype control (IC) (anti-ovalbumin, HYB099-01; Statens Serum Institut, Copenhagen, Denmark) for 30 min at room temperature before seeding cells. The non-migrated cells were removed using a cotton bud. The cells that had migrated on the underside of the membrane were stained using Hemacolor (Sigma-Aldrich) and counted in 4 fields of each filter using Olympus IX73 microscope with ×20 objective.

CCl₄ induced liver fibrosis in rats

### Animal Experiments

Male Sprague Dawley rats (200-225 g) were obtained from Janvier labs and were subjected to 12-hour light dark cycle and provided free access to water and food. Liver fibrosis was induced by carbon tetrachloride (CCL₄) (Sigma)+ phenobarbital intoxication. Two weeks prior to CCL₄ treatment, 35 mg/dl phenobarbital is added to the rats' drinking water. Then phenobarbital administration was stopped. Thereafter rats were administrated CCL₄ in olive oil or olive oil alone once per week by oral gavage. The first dose of CCL₄ in olive oil was 412 mg/Kg (Day 0). In order to reduce the mortality rates the following doses of CCL₄ were adjusted based on body weight change from the previous week as showed in Table 1. At day 20, rats received their first anti-MFAP4 antibody hAS0326 Fc-neutralized (FC-neutralized as described in Schlothauer et al., 2016) dose, 20 mg/kg intravenously. Thereafter they received 2 doses of 5 mg/kg of anti-MFAP4 at day 27 and 34. At day 38 rats were euthanized, livers and spleens were excised in 1 piece and weighed. Liver specimens from the left and right lobes were either fixed in formalin for immunohistochemical analysis or snap frozen in liquid nitrogen for further analysis such as hepatic hydroxyproline determination, RNA and protein extraction.

TABLE 1
CCL₄ treatment scheme

| Weekly weight change | Dose administered |
|---|---|
| Increase 0-5 g | Same as previous dose |
| Increase 6-10 g | 125% of previous dose |
| Increase >10 g | 150% of previous dose |
| Decrease 1-5 g | 75% of previous dose |
| Decrease 6-10 g | 50% of previous dose |
| Decrease >10 g | No dose |

Protein Extractions from Liver Tissues

Frozen tissues were grinded in liquid nitrogen and incubated in PBS+10 mM EDTA+Protease and Phosphatase inhibitors (Roche), followed by centrifugation (10000 g for 10 minutes). The supernatant was collected, and total protein amount was determined by Bradford assay (Biorad). 0.1 mg/ml of total liver protein was taken from each sample to measure MFAP4 level by ELISA.

### Detection of MFAP4 Level in Serum and Liver Tissues by ELISA

96 well MaxiSorb plates were coated with 1 μg/ml Anti-MFAP4 antibody HYB 7-18 by incubation overnight at 4° C. in PBS. Each step was followed by four rounds of washing in PBS 0.05% Tween 20. The washing buffer was also used for blocking by incubation for 1h at RT. The following day, 100 μl/well of the samples and standard diluted in wash buffer were added to the plate and incubated for 2 h at room temperature on orbital shaker (300 rpm). Thereafter, 0.5 μg/ml biotinylated anti-MFAP4 HYB 7-14 in washing buffer was added and incubated for 1h at RT with shaking. Streptavidin-conjugated horseradish peroxidase (Invitrogen) was diluted 1:2000 in washing buffer and incubated for half an hour at RT. Finally, O-Phenylenediamine Dihydrochloride (OPD) (Thermo Fisher Scientific) dissolved in enzyme substrate buffer for HRP+H₂O₂ (added prior to use) was added to the plate and allowed to react for 15 minutes in dark at room temperature. Then 100 μl/well of 1M H₂SO₄ was added to the plate to stop the color development. Optical density was read at 492 nm wavelength with 620 nm as reference.

Isolation of mRNA, Reverse Transcriptase and Real Time PCR

Total RNA was extracted from frozen liver tissue grinded with liquid nitrogen by incubation with Trizol reagent (Sigma). Total RNA was reverse transcribed to complementary DNA using (Thermo Fisher Scientific) according to the manufacturer's instructions. Gene expression was tested by real-time PCR using the following TaqMan probes all obtained from Thermo Fisher Scientific: COL1A1 Rn01463848_m1, ACTA2 Rn01759928_g1, IL1P Rn00580432_m1, TGFβ1 Rn00572010_m1, TIMP1 Rn01437681_m1, MFAP4 Rn1452830_g1. The relative expression levels were normalized to Tbp Rn01455648_m1.

### Example 2—MFAP4 and Organ Fibrosis

### Aim

To study the presence of MFAP4 in fibrous tissue.

### Materials and Methods

See example 1

### Results

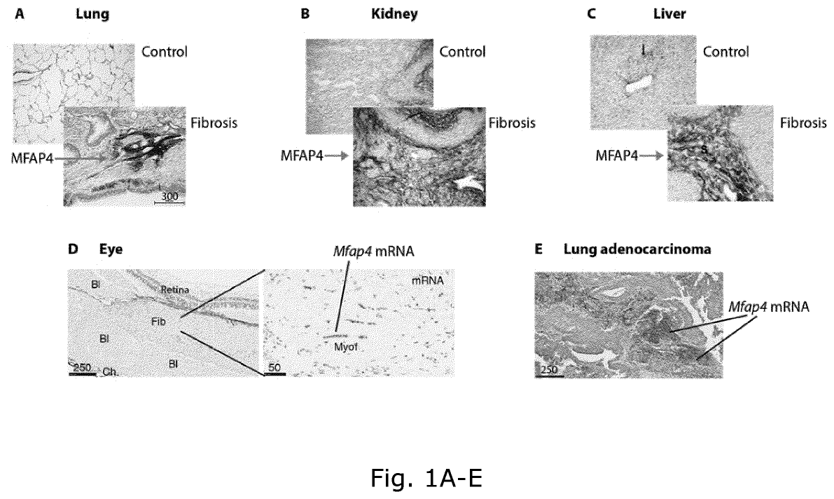

FIG. 1 demonstrates using immunohistochemistry that the MFAP4 accumulates in diverse types of organ fibrosis like lung, kidney and liver (FIG. 1A-C).

MFAP4 is synthesized by the myofibroblast, which is a central fibrotic cell type. This is e.g. demonstrated in FIG. 1D-E by in situ hybridization. A representative sample of a human eye with subretinal fibrotic deposition (FIG. 1D) show Mfap4 mRNA expression in myofibroblasts. Similarly, this is demonstrated in lung adenocarcinoma (FIG. 1E).

### Conclusion

The myofibroblast activation is at the very base of the fibrotic process. These data demonstrate an accumulation of MFAP4 in fibrous tissue and in the myofibroblast.

### Example 3—Reversibility of Phenotype in ARPE19 Cells Using Anti-MFAP4

### Aim

To study the effect of treatment with anti-MFAP4 on myofibroblast-like cells from retina (ARPE19).

### Materials and Methods

See example 1

### Results

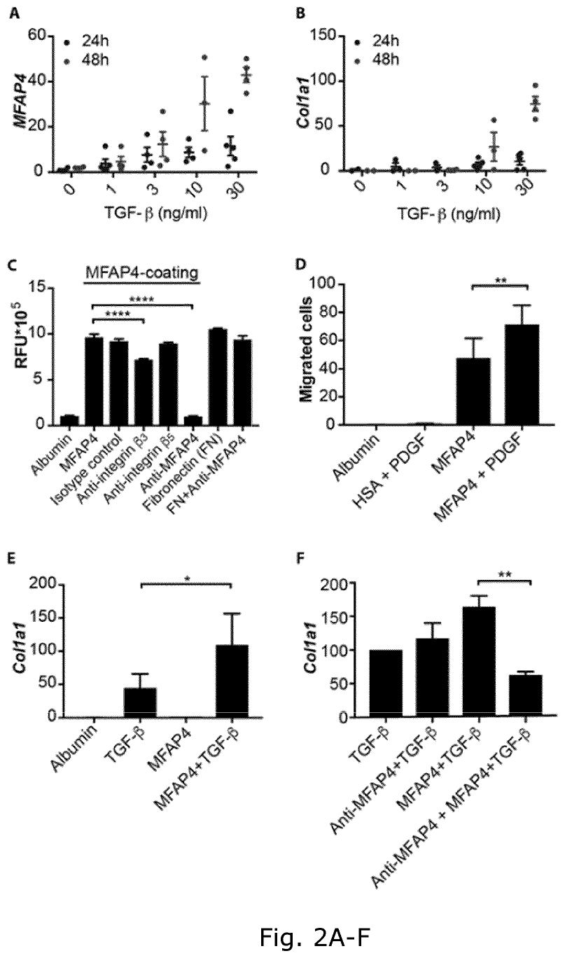

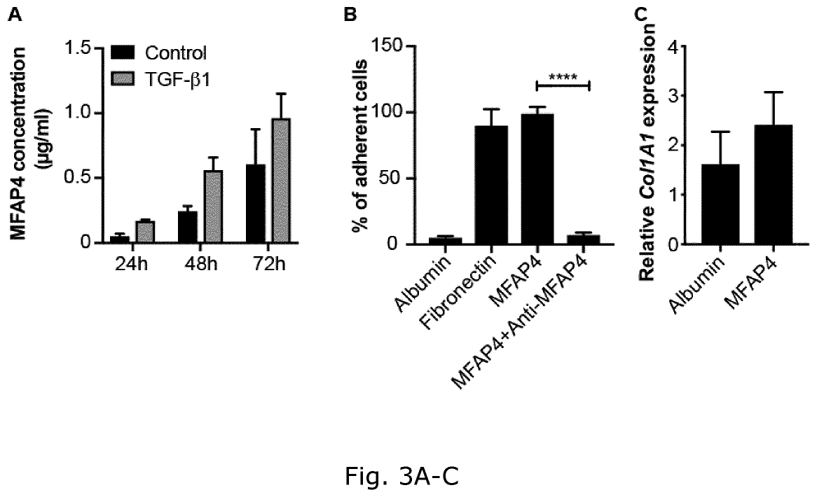

MFAP4 synthesis can be induced in vitro in myofibroblast-like cells derived from different organs. TGF−-induction of retinal pigment epithelial cells (ARPE19) trans-differentiation resulted in increased synthesis of both MFAP4 (MFAP4) mRNA (FIG. 2A) and Col1a1 (Collagen type 1) mRNA (FIG. 2B).

MFAP4 directly causes activation of these cells in terms of adhesion (FIG. 2C), migration with and without PDGF-enhancement (FIG. 2D) and collagen type 1 synthesis (FIG. 2E) when they are seeded on MFAP4-coated surface. Data in D and E are independent of MFAP4s role in adhesion.

Treatment with anti-MFAP4 shows that this activation can be reversed (FIGS. 2C and F).

### Conclusion

MFAP4 appears to be up-regulated by myofibroblast activity when the ARPE-19 cells are transformed into myofibroblasts. This transformation produces migratory myofibroblasts, which may infiltrate the injured regions and lay down collagenous septa as the collagen production is also shown to increase. However, the results demonstrate that treatment with anti-MFAP4 reverses this phenotype including decreasing the production of collagen type I. Thus, demonstrating that anti-MFAP4 may prevents formation of fibrous tissue.

### Example 4—Reversibility of Phenotype in Primary Hepatic Stellate Cells Using Anti-MFAP4

### Aim

To study the effect of treatment with anti-MFAP4 on myofibroblast-like cells from the liver.

### Materials and Methods

See example 1

### Results

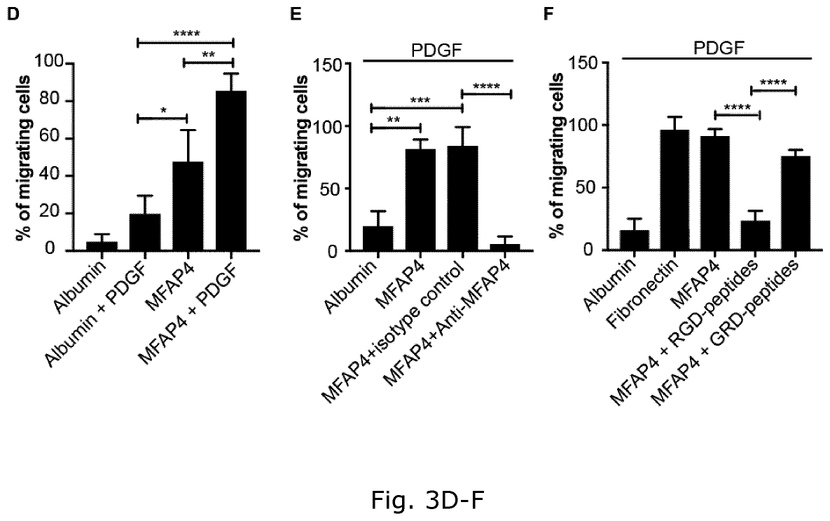

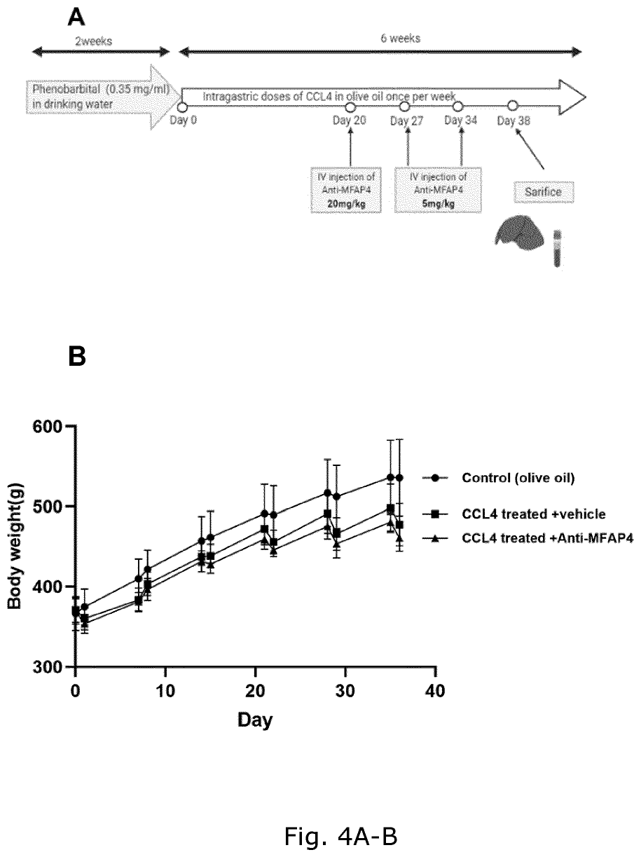

MFAP4 synthesis was likewise induced in primary hepatic stellate cells (FIG. 3A). When these cells are grown in vitro, they spontaneously trans-differentiate into myofibroblast-like cells and differentiation may be further enhanced with TGF-treatment. In line with this, MFAP4 synthesis was enhanced both by time in culture and by TGF-treatment.

Furthermore, MFAP4 directly causes activation of these cells in terms of adhesion (FIG. 3B). Also, a tendency for enhanced collagen type 1 synthesis was observed when the cells were seeded on MFAP4-coated surface (FIG. 3C). Moreover, MFAP4 increased cellular migration with and without PDGF-enhancement (FIG. 3D-F). Data in C-F are independent of MFAP4s role in adhesion.

The migratory activation was reversible with anti-MFAP4 treatment (FIG. 3E).

Also, it was demonstrated that the effect was mediated through RGD-dependent integrin as shown by specific blocking of migration with competing RGD-peptides and not with RGD-peptides (FIG. 3F).

### Conclusion

MFAP4 appears to be up-regulated by myofibroblast differentiation and activation, when primary liver cells differentiate into myofibroblast-like cells. This transformation produces migratory myofibroblasts, which may infiltrate the injured regions and lay down collagenous septa as the collagen production is also shown to increase. However, the results demonstrate that treatment with anti-MFAP4 reverses this phenotype including decreasing the production of collagen type I. Thus, demonstrating that anti-MFAP4 may prevent formation of fibrous tissue.

### Example 5—Treatment of Induced Liver Fibrosis in Rats with Anti-MFAP4

### Aim

To study the effect of treatment with anti-MFAP4 on liver fibrosis.

### Materials and Methods

See example 1

### Results

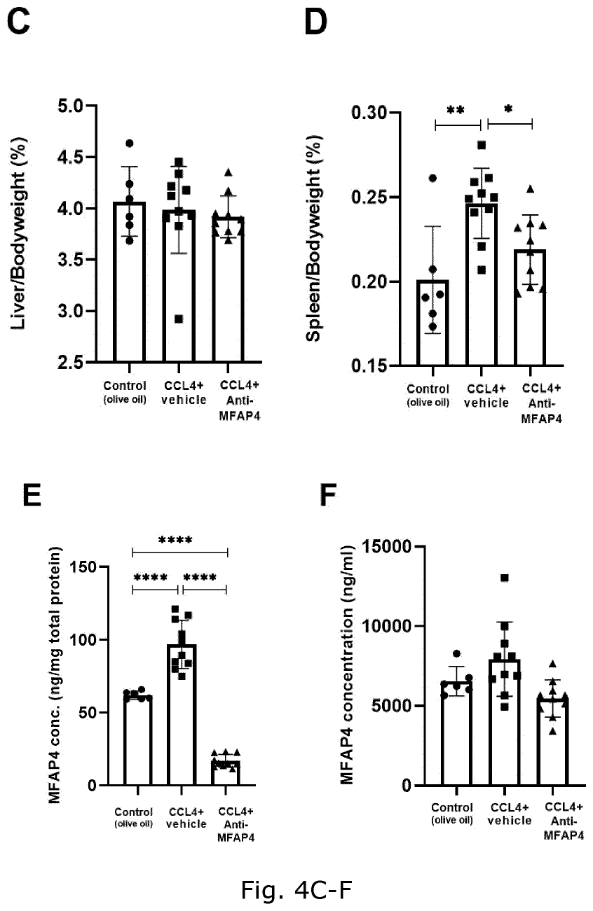

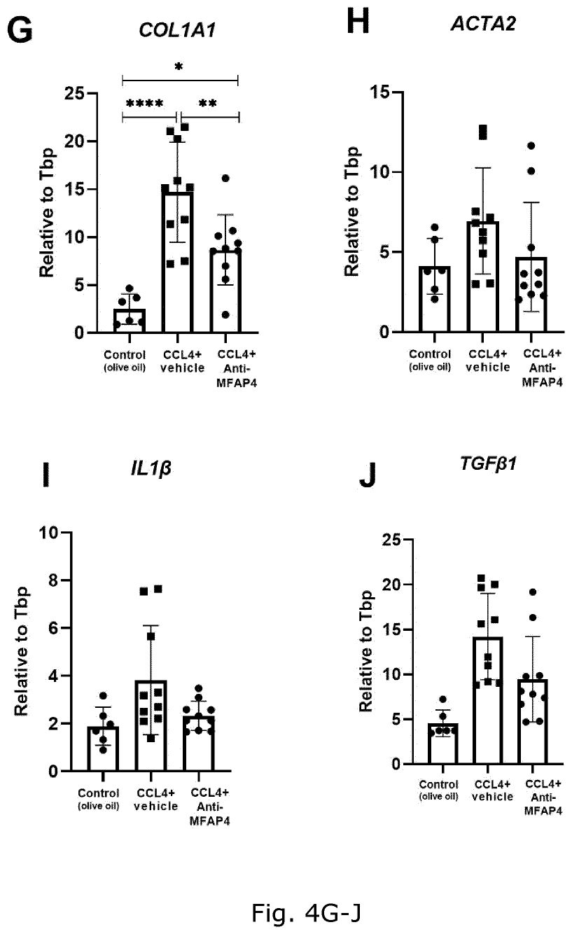

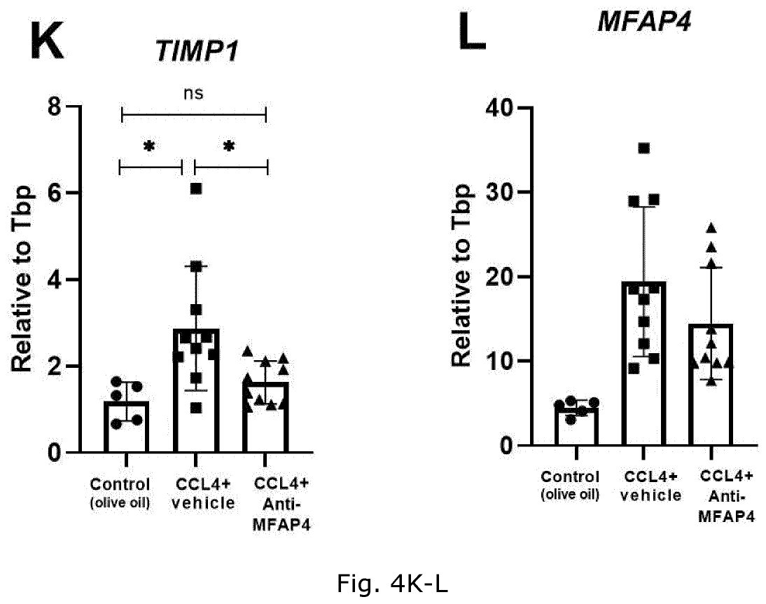

Rat liver fibrosis was induced by two weeks phenobarbital in drinking water succeeded by 6 weeks carbontetrachloride (CCl₄) treatment by oral gavage. The rats received a total of three IV doses of anti-MFAP4 or vehicle treatment on days 20, 27 and 34, before they were sacrificed on day 38 (FIG. 4A). This resulted in a reduced rat body weights but not liver-to-body weight ratio but significantly increased the spleen-to-body weight ratio (proxy for portal hypertension) and this effect was significantly reduced by anti-MFAP4 treatment (FIG. 4B-D).

Immunodetection of MFAP4 was significantly induced in the model liver tissue and significantly reduced in this tissue (FIG. 4E), while insignificantly reduced in (FIG. 4F) blood after anti-MFAP4 treatment. Relative mRNA expression of fibrotic and inflammatory markers: collagen type 1 (Col1A1) mRNA (FIG. 4G), alpha-smooth muscle actin (ACTA2) (FIG. 4H), IL-1b (IL1b) (FIG. 4I), TGF-1 (TGFb1) (FIG. 4J), TIMP-1 (TIMP1) (FIG. 4K), and MFAP4 (MFAP4) (FIG. 4L) mRNA was induced in rat liver tissue by the model and reduced with anti-MFAP4 treatment.

### Conclusion

These data demonstrate that treatment of rats having induced liver fibrosis show less Collagen type I after treatment indicating decrease in the formation of fibrous tissue.

The present data thus characterizes a novel and unique mechanism whereby a therapeutic monoclonal antibody (Mab) is densely fixed in fibrotic depositions, thereby detaching cells and shielding essential mechanosensing, activating cues from the extracellular matrix (ECM) microenvironment for a central cell in pathology; the myofibroblast.

The advantage of this novel approach will be the resolution of myofibroblast activation despite of ongoing activation signalling from growth factor and cytokine expression. The myofibroblast activation is at the very base of the fibrotic process as a whole. Transformation produces migratory myofibroblasts which infiltrate the injured regions and lay down collagenous septa. MFAP4 appears to be up-regulated by myofibroblast activity in various types of organ fibrosis. Therefore, the novel anti-MFAP4 Mab has a potential suitability to multivalent etiology of fibrosis.

### Example 6—Anti-MFAP4 inhibits experimental liver fibrosis

### Aim

To study the role of MFAP4 in modulation of HHSteCs biology and further to study the efficacy of anti-MFAP4 on inhibition of experimental liver fibrosis.

### Material and Methods

### Standard Buffers

EDTA: 0.5M, Na₂EDTA 2H₂O (6M gel); 10 g NaOH (Merck) pH 8. PBS: 8 mM Na₂H₂PO₄, 1.5 mM KH₂PO₄, 140 mM NaCl, 0.003 mM KCl, pH 7.4. PBS/tween: PBS with 0.05% tween 20. Substrate buffer for Horse radish peroxidase (HRP): 35 mM citric acid, 67 mM Na₂HPO₄, pH 5.0.

### Cell Culture

Primary human hepatic stellate cells (HHSteC, 5300, ScienCell) were cultured in stellate cell medium (SteCM, 5301, Sciencell) supplemented with 2% fetal bovine serum (FBS, 0010, ScienCell), 1% Stellate cell growth supplement (SteCGs, 5352, Sciencell) and 1% penicillin-streptomycin (P/S, 0503, ScienCell) and maintained at 37° C. at 5% CO₂. Cells were passaged when 80-90% confluent and experiments were carried between passage 3 and 10. Prior to MFAP4 and/or TGFβ1 stimulation, cells were starved in SteCM with 0% FBS and 0% SteCGs for 16 hours. For MFAP4 stimulation, 6 well plates were coated with 10 μg/ml recombinant human MFAP4 (rhMFAP4) or human serum albumin (HSA, Sigma-Aldrich) in PBS1× (Sigma-Aldrich) overnight at 4° C. The following day, wells were washed and blocked with 10 mg/ml of HSA for 1h. Then, HHSteC were seeded at a density of 200,000 cells/well in SteCM with 0.5% FBS+/−TGFβ1 (5 ng/ml, 240-BO10, R&D systems) for 72 hrs, then mRNA was isolated for assessing gene expression. For checking MFAP4 expression by HHSteC upon activation, HHSteC were seeded at a density of 200,000 cells/well and stimulated with TGFβ1 (5 ng/ml) in SteCM media supplemented with 0.5% for 24, 48 and 72 h. For each time point, cell supernatant was collected for detection of MFAP4 expression by ELISA and mRNA was isolated for assessing gene expression.

### Adhesion Assay

Black 96-well Maxisorp FluroNunc™ microtiter plates (Nunc) were coated, at 4° C. overnight, with 10 μg/ml rhMFAP4, fibronectin (FN, Sigma-Aldrich) or HSA in PBS. The following day, plates were washed and blocked for 1h with 10 mg/ml HSA. For blocking MFAP4, immobilized rhMFAP4 was incubated for 1h at room temperature with 20 μg/ml of anti-MFAP4 antibody (hAS0326) before seeding cells. For studying the involvement of RGD motif and integrins in MFAP4-cell interaction, HHSteCs were incubated with 100 μg/ml of synthetic GRGDS or SDGRG peptides (Sigma-Aldrich) or 10 μg/ml of anti-integrin αᵥβ₃ (cloneLM609, Merck), anti-integrin αᵥβ₅ (clone P1F6, Merck) or isotype control (IC, anti-ovalbumirn HYB099-01; Statens Serum Institute, Copenhagen, Denmark) for 30 minutes at room temperature. Cell adhesion was quantified using Vybrant cell adhesion kit (Molecular probes, Invitrogen). Briefly, cells were detached using standard trypsinization and incubated in serum free medium containing Calcein AM dye (5 μM for 5 million cells/ml) for 30 minutes at 37° C. Then cells were washed twice with PBS and seeded at a density of 100,000 cells/well. After 1h of incubation at 37° C., plates were washed and 200 μl/well of PBS was added. Fluorescence was measured using Victor™ 3 plate reader (Perkin Elmer) (λ excitation=485 nm, λ emission=535 nm).

### Migration Assay

Cell migration assay was performed using transwell inserts with polyester filter membrane of 8 μm pore-size (Corning). The underside of the filters was coated overnight at 4° C. with 10 μg/ml rhMFAP4 or HSA in PBS. HHSteC were seeded in the transwell inserts upper chamber in serum free medium at a density of 50,000 cells/insert and were allowed to migrate for 3h towards the lower chamber containing serum free medium+/−Platelet derived growth factor-BB (PDGF) (100 ng/ml, R & D systems). For MFAP4 blocking experiments, 10 μg/ml of anti-MFAP4 antibody (hAS0326) or IC were added to the lower chamber. For studying the role of RGD motif and integrins in MFAP4-mediated cells migration, HHSteC were incubated with 100 μg/ml of synthetic GRGDS and SDGRS peptides or 10 μg/ml of anti-integrin αᵥβ₃ or IC, for 30 minutes at room temperature before seeding cells. After 3 h, the non-migrated cells on the upper side of the filter were removed using a cotton bud. The migrated cells on the underside of the filter were fixed and stained using Hemacolor staining solution 1, 2 and 3 added successively (111955, 111956, 111957, Merck), and filters were left to dry overnight. The next day, cells were counted in 4 different fields of each filter using Olympus IX73 microscope, with 20× objective.

### Proliferation Assay

Proliferation of cells was assessed using WST-1 assay. 96-well tissue culture plates (Nunc) were coated at 4° C. overnight with 10 μg/ml rhMFAP4 or HSA. HHSteC were serum starved for 16 hours in SteCM media. The next day, plates were washed and blocked with 10 mg/ml HSA for 1h at room temperature. Cells were seeded at a density of 14,000 cells/well in STeCM media with 0.5% FBS and incubated for 4, 24, 48 or 72 h at 37° C. 10p of cell proliferation reagent WST-1 (Sigma-Aldrich)/100 μl culture medium was added to the wells at each timepoint. After 2h of incubation at 37° C., formazan dye (product of tetrazolium salt WST-1 cleavage) is quantified by measuring the absorbance at 450 nm with a reference wavelength of 690 nm using ELISA microplate reader (TECAN).

### Rat Model of Liver Fibrosis

Male Sprague Dawley rats (200-225 g) were obtained from Janvier labs and were subjected to 12 hours light dark cycle and provided free access to water and food ad libitum. Liver fibrosis was induced using phenobarbital+carbon tetrachloride (CCl₄) intoxication.

Two weeks prior to CCl₄ treatment, rats received 0.35 mg/ml of phenobarbital in drinking water. Thereafter, rats were administered CCl₄ (Sigma) in olive oil (Sigma-Aldrich) once per week for 6 weeks by oral gavage. The first dose of CCl₄ was 412 mg/kg (Day 0). In order to reduce the mortality rates, the following doses of CCl₄ were adjusted based on the body weight change from the previous week. Control group received olive oil by oral gavage once per week for 6 weeks and no phenobarbital. At day 20, CCl₄ and phenobarbital treated rats received their loading dose of anti-MFAP4 antibody hAS0326 Fc-neutralized (FC-neutralized as described in Schlothauer et al., 2016) or vehicle control (10 mM Histidine 10% Trehalose), by tail vein intravenous injection. Thereafter they received two subsequent maintenance doses at day 27 and 34 of anti-MFAP4 antibody hAS0326 Fc-neutralized or vehicle control. At day 38, rats were sacrificed by CO₂ overdose.

### Validating Mouse Model of Liver Fibrosis

Male (C57Bl6/J) mice (12 weeks old) were purchased (Charles River Laboratories Research Model and Services Germany, Sulzfeld, Germany). The animals were kept at 22° C. with a 12:12-h day-night cycle in individually ventilated cages. Liver injury was induced by inhalative CCl₄ exposure for 7 weeks (one time a week for the first 4 weeks followed by intoxications two times a week for the next 3 weeks). Briefly, CCl₄ was insufflated with a flow of 2 l/min for 1 min, the cage remained closed for another minute, and CCl₄ was finally removed under the hood for 10 min. CCl₄ was supplemented by a high-fat cholesterol-rich diet (WD; Ssniff), or CCl₄ was administered alone. Water and chow were provided ad libitum. All animals intoxicated with CCl₄ additionally received phenobarbital (0.33 g/l) via drinking water as an inducer of the cytochrome P-450 metabolic activity. Control age-matched untreated mice were used in all experiments. At week 5, 20 CCl₄ treated mice received two loading doses of anti-MFAP4 antibody hAS0326 Fc-neutralized (FC-neutralized as described in Schlothauer et al., 2016) or vehicle control (10 mM Histidine 10% Trehalose), by tail vein intravenous injection. Thereafter, they received two subsequent maintenance doses both week 6 and week 7 of anti-MFAP4 antibody hAS0326 Fc-neutralized or vehicle control. Before being euthanized, mice received ketamine-xylazine anesthesia (100 mg ketamine/kg body wt and 10 mg xylazine/kg body wt) that was injected intraperitoneally. Liver samples were fixed in formaldehyde (4%) and subsequently embedded in paraffin or fixed in Tissue Tek OCT, respectively (Sakura Finetek Germany, Staufen, Germany). This test was performed by the independent laboratory of Professor Jonel Trebicka, Goethe-Universitat Frankfurt am Main, Medical Department I, Germany.

### Blood and Tissue Collection

From rat animal model, blood was collected by heart puncture. Liver and spleen were excised in one piece and weighed. Liver specimens from the left and right lobes were either fixed in formalin for immunohistochemical analysis or snap frozen in liquid nitrogen for further molecular analysis.

### Preparation of Liver Homogenate and Protein Extraction

Frozen livers were grinded in liquid nitrogen until obtaining a powder, 50-100 mg of the grinded tissue were transferred to a fresh Eppendorf tube and homogenized in PBS+10 mM EDTA containing protease and phosphatase inhibitors (Roche), followed by centrifugation for 10,000 g for 10 minutes. The supernatant was collected, and the total protein concentration was determined using DC™ protein assay (Bio-rad) according to the manufacturer's instructions.

### Measurement of MFAP4 by ELISA

MFAP4 expression is serum, liver homogenate and HHSteCs supernatant was measured using sandwich ELISA. 96-well Maxisorb microplates (Nunc) were coated with 1 μg/ml of anti-MFAP4 antibody HG-HYB7-18 (for detection of rats MFAP4)/HG-HYB 7-5 (for detection of human MFAP4) at 4° C. overnight. The next day, plates were washed and blocked with PBS/tween for 1h at room temperature. 100 μl/well of samples and standard (rhMFAP4) diluted in PBS/tween were added to the plate and incubated for 2 h at room temperature on orbital shaker. Thereafter, 0.5 μg/ml of biotinylated anti-MFAP4 antibody HG-HYB 7-14 (for rats MFAP4)/HG-HYB 7-18 (for human MFAP4) diluted in PBS/tween was added to the plate and incubated for 1h at room temperature with shaking. Then, plates were successively incubated with horseradish peroxidase (HRP)-conjugated streptavidin (1:2000, diluted in PBS/tween, Invitrogen) for half an hour at room temperature, O-phenylenediamine dihydrochloride (OPD) (0.4 mg/ml, H1009, Sigma) dissolved in HRP with 0.03% H₂O₂, for 15 min at room temperature in the dark. Finally, 100 μl/well of 1M H₂SO₄ was added to stop the color development. Optical density was read at 492 nm wavelength with 620 nm as reference using ELISA microplate reader.

### Measurement of Hydroxyproline in Rat Liver

Hydroxyproline (HYP) was measured using hydroxyproline colorimetric assay kit (K555, Biovision) according to the manufacturer's instructions, from 2 snap frozen pieces of the left and right lobe.

### Measurement of Liver Enzymes

Alanine aminotransferase (ALT) and aspartate aminotransferase (AST) activities in rat's serum were measured using commercial Kit according to the manufacturer's instructions (MAK052 for ALT; MAK055 for AST, Sigma)

### Quantitative Real Time PCR

Total RNA was extracted from homogenized liver tissue (from 2 snap frozen pieces of the left and right lobe) or from HHSteC using TRIzol reagent according to the manufacturer's instructions. 1-2 μg of RNA was reversed transcribed to complementary DNA (cDNA) using High-capacity reverse transcriptase kit (Thermo Fisher). Relative gene expression was quantified by Real time PCR using TaqMan universal PCR master mix (Invitrogen) and TaqMan gene expression assays (Thermo Fisher) represented in Table 2.

TABLE 2
TaqMan assay used for qPCR

| Species | Gene | Taqman assay |
|---|---|---|
| Rat | Col1A1 | Rn01463848_m1 |
| | ACTA2 | Rn01759928_g1 |
| | TGFβ1 | Rn00572010_m1 |
| | TIMP1 | Rn00587558_m1 |
| | MMP2 | Rn01538170_m1 |
| | MFAP4 (LOC1025) | Rn01452830_g1 |
| | IL1β | Rn00580432_m1 |
| | Col3A1 | Rn01437681_m1 |
| | Tbp | Rn01455648_m1 |
| Human | Col1A1 | Hs00164004_m1 |
| | ACTA2 | Hs00426835_g1 |
| | MFAP4 | Hs00412974_m1 |
| | Tbp | Hs99999910_m1 |

### Immunohistochemical Analysis

Liver tissues were fixed in 10% neutral buffered formalin for 24 h, then transferred to PBS1× containing 0.05% of sodium azide (52002, Sigma). Paraffin embedded tissue blocks were cut in 4 μm thick sections, dried at 60° C., deparaffinized in xylene and hydrated in graded ethanol. Reactivity of endogenous biotin was blocked with 1.5% hydrogen peroxide.

Different antigen retrieval techniques were performed followed by the incubation with the corresponding primary antibodies against MFAP4, −SMA, CD11b and CD31. Details of the antibodies and antigen retrieval techniques used were specified in Table 3. For FITC-conjugated antibodies, a polyclonal rabbit anti-FITC secondary antibody (P5100, DAKO) was used and visualized with goat anti-rabbit HRP labelled antibody (Envision+ K4003, DAKO). For CD11b staining, goat anti-rabbit HRP labelled antibody (Envision+ K4003, DAKO) was used as secondary antibody. CD31 and anti-MFAP4 antibody (hAS0326 Fc-neutralized) staining were visualized using OmniMap anti-rabbit-HRP and OmniMap anti-Goat-HRP detection system (Ventana Medical Systems) respectively, automated at the Discovery Ultra immunostainer (Ventana Medical systems). Collagen staining was performed using Sirius red.

TABLE 3
List of primary antibodies, dilution and Antigen retrieval
techniques used for immunohistochemistry

| | | Working | |
|---|---|---|---|
| Target | Antibody | solution | Antigen Retrieval |
| MFAP4 | FITC-conjugated Anti- | 1:200 | Protease treatment |
| | MFAP4 (HG-HYB 7- | | (0.05% protease type |
| | 14) | | XIV; Sigma) |
| A-SMA | FITC-conjugated | 1:1000 | Microwave heating |
| | anti- - SMA (Clone 1A4, | | with Tris-EGTA buffer |
| | Sigma) | | (TEG-buffer) |
| CD11b | Recombinant Anti- | 1:10000 | Microwave heating |
| | CD11b [EPR1344] | | with Tris-EGTA buffer |
| | (Ab133357, Abcam) | | (TEG-buffer) |
| CD31 | Recombinant Anti- | 1:100 | CC1_32_100 |
| | CD31 [EPR17259] | | |
| | (Ab182981, Abcam) | | |
| hAS0326 | Goat anti-Human IgG Fc | 1:2000 | CC1_32_100 |
| | (Ab97221, Abcam) | | |

FITC: Fluorescein isothiocyanate, TEG-buffer: 10 mM Tris; 0.5 mM EGTA, pH 9.0; CC1: Cell conditioning solution 1 (pH 8.5; Ventana Medical Systems), CC1_X_X: CC1_minutes incubated_degree Celsius.

### RNA In Situ Hybridization

RNA in situ hybridization was performed on rat liver tissue using a modified version of the RNA scope 2.5 high-definition procedure. Briefly, 4 μm sections were in situ hybridized with 20 ZZ probe pairs (Advanced Cell Diagnostics, ACD) covering nucleotide in the region between 366-1417 of rat MFAP4 mRNA (NM_001034124.2) followed by thiamine signal amplification and visualized with Liquid permanent red (K0640, Agilent).

### Image Acquisition and Quantification

Immunohistochemical sections were scanned at 40× magnification using NanoZoomer-XR (Hamamatsu photonics; Hamamatsu City, Japan), and image acquisition was performed using NDP.view2 software (NanoZoomer Digital Pathology; Hamamatsu Photonics). The contrast of the representative images was automatically adjusted in Adobe Photoshop 2021. Sirius red quantification was performed in 8 random non-overlapping fields per slide (pictures were obtained using NDP.view2 software at 2.5× magnification). −SMA quantification was performed in 9-11 random non-overlapping fields per slide (pictures were obtained using NDP.view2 software at 2.5× magnification), CD11b quantification was performed in 7-9 random non-overlapping fields per slide (pictures were obtained using NDP.view2 software at 5× magnification), Sinusoid capillarization was assessed by quantification of CD31 immunohistochemical staining in 15-17 random non-overlapping fields per slide in the parenchymal area of liver lobules without including the portal areas (pictures were obtained using NDP.view2 software at 20× magnification). Sirius red, −SMA, CD11b and CD31 positive area was calculated with ImageJ software, using the threshold technique, as the percentage of pixels above the threshold value adopted in respect to the total pixels in the corresponding area.

### Statistical Analysis

Statistical analysis was performed in prism 8 Software. Data are presented as mean+SEM. Normality of the data was checked using Shapiro-Wilk test and QQ plot. Parametric results were analyzed using Student's t test, one-way or two-way analysis of variance (ANOVA) followed by Tukey's multiple comparisons test. Non-parametric results were analyzed using Mann-Whitney test or Kruskal-Wallis test.

### Results

Mfap4 Mediates HHSteCs Adhesion and Migration In Vitro Through RGD-Integrin αvβ3 Dependent Mechanism

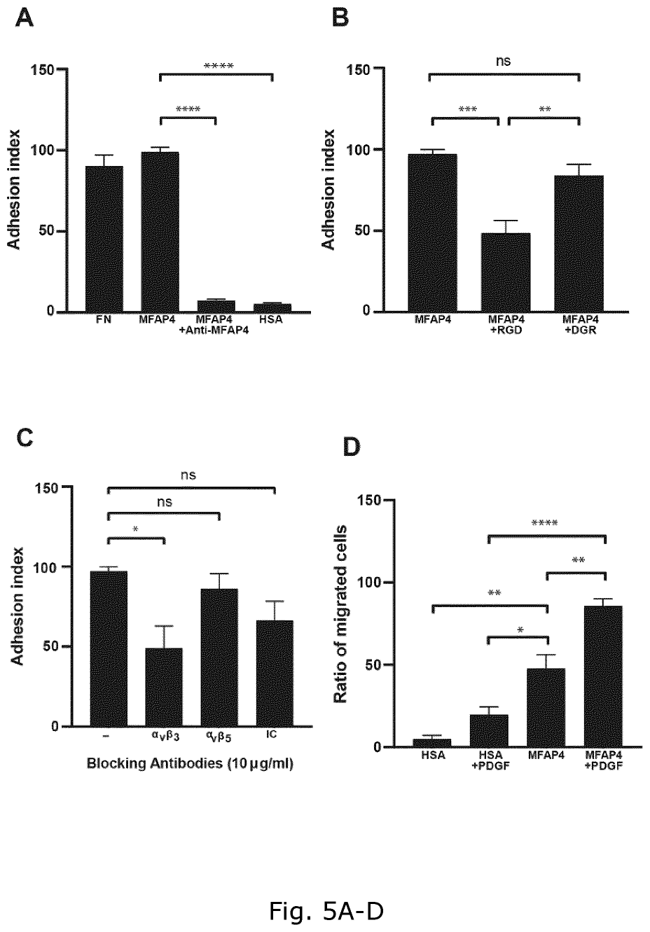

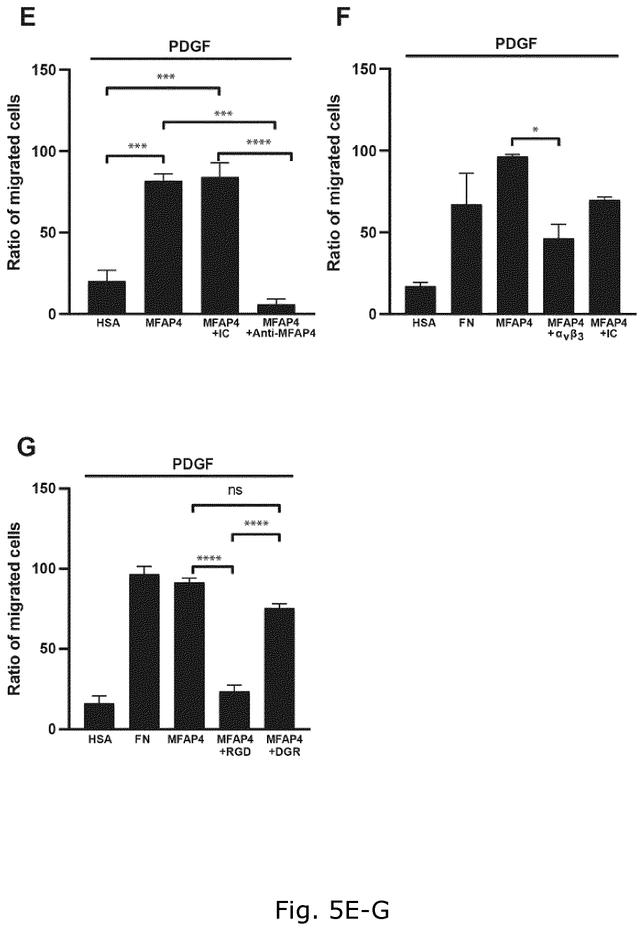

Fluorescent based adhesion assay revealed that HHSteCs adhere to immobilized rhMFAP4 to the level of the positive control fibronectin within 1h whereas no adhesion was detected on HSA (negative control). In addition, blocking MFAP4 using specific humanized anti-MFAP4 antibody, hAS0326, inhibited completely the attachment of HHSteC to rhMFAP4 (FIG. 5A). To evaluate the involvement of MFAP4-RGD motif, an integrin ligand, in MFAP4 and HHSteC interaction, cells were preincubated with synthetic RGD peptides, which blocks the extracellular domain of RGD dependent cellular integrins, or with its counterpart DGR peptides as negative control. Cellular adhesion to rhMFAP4 was significantly decreased in the presence of RGD peptides, but not control DGR (FIG. 5B).

Preincubation of HHSteC with anti-integrin αvβ3 inhibited cellular attachment to rhMFAP4, whereas no significant reduction in cells adhesion was observed in the presence of anti-integrin αvβ5 blocking antibodies (FIG. 5C). Suggesting that integrin αvβ3 is the main MFAP4 receptors on HHSteC.

To evaluate HHSteC migratory response to MFAP4 stimulation, a migration assay was performed using transwell inserts. Results showed that the 3 hours migration of HHSteC was increased significantly on MFAP4 coated inserts relative to HSA in the presence or absence platelet-derived growth factor-BB (PDGF-BB) (FIG. 5D) as chemoattractant for HHSteCs. Furthermore, anti-MFAP4 antibody completely abolished the migration of HHSteC towards MFAP4 (FIG. 5E). This mechanism of cell migration towards a gradient of immobilized molecules is known as haplotaxis. To further understand MFAP4-induced haptotaxis towards HHSteC and define the components involved in this mechanism, cells were incubated with synthetic RGD peptides or anti-integrin αvβ3 blocking antibodies which reduced significantly the migratory response of HHSteC to MFAP4 stimulation (FIG. 5F-G). Suggesting that MFAP4 interaction with HHSteC through RGD-integrin αvβ3 binding is required for MFAP4-derived HHSteC haptotaxic activity.

### MFAP4 is Synthesized by HHSteCs Upon TGFβ1 Stimulation and Enhances the Profibrotic Effect of TGFβ1 on HHSteCs In Vitro

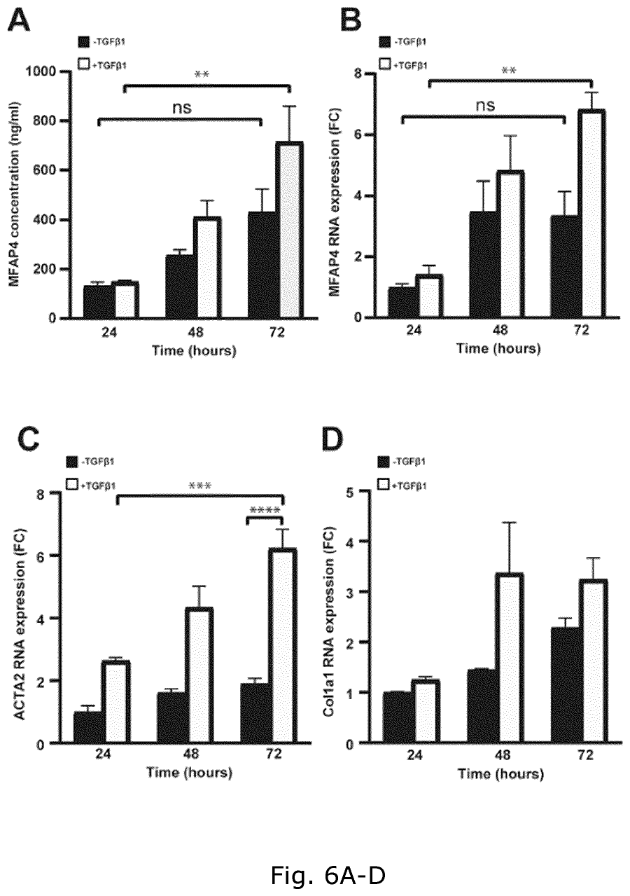

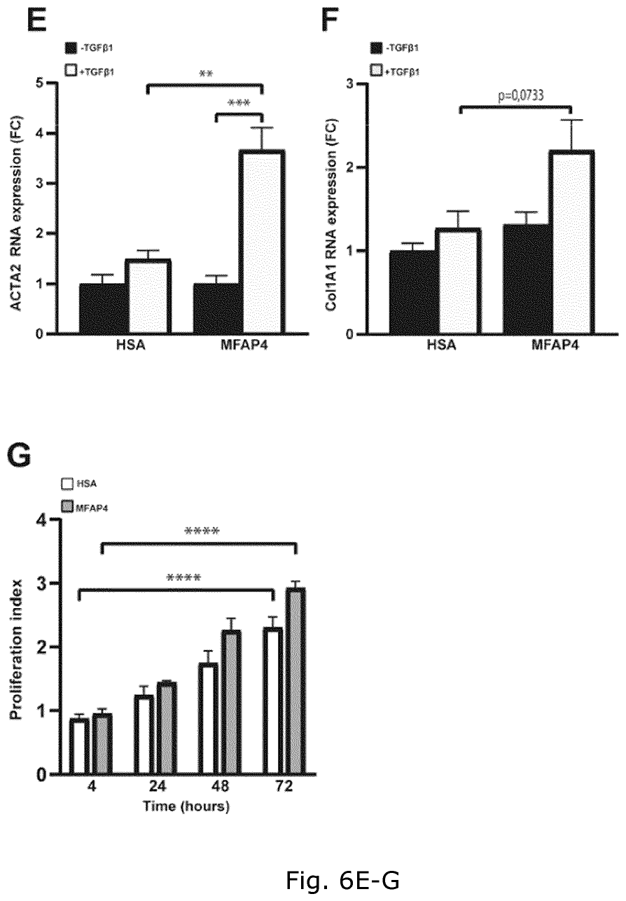

MFAP4 synthesis in HHSteC was significantly upregulated in TGFβ1 stimulated cells, in a time dependent manner in parallel with increased −SMA and type 1 collagen expression representing the activation of the cells (FIG. 6A-D).

To investigate the effect of MFAP4 on HHSteCs transdifferentiation, HHSteC were on MFAP4 or HSA (negative control) coated wells in the presence or absence of TGFβ1 stimulation. In the absence of TGFβ1, MFAP4 failed to induce HHSteCs activation, whereas in the presence of TGFβ1, MFAP4 enhanced HHSteCs activation featured through upregulation of ACTA2 and Col1A1 compared to TGFβ1 stimulated cells on HSA, suggesting that TGFβ1 and MFAP4 exert a synergetic effect on HHSteCs activation in vitro (FIG. 6E-F).

To explore MFAP4 effect on HHSteCs proliferation, cells were seeded onto rhMFAP4 and allowed to proliferate for 24, 48 or 72h. HHSteC proliferation increased significantly over time however it was not significantly different from the proliferation pattern of cells seeded on HSA for each of the corresponding time point, suggesting that MFAP4 is not a crucial inducer of activated HHSteCs proliferation (FIG. 6G).

Expression of MFAP4 Increases in Rat Model of Liver Fibrosis and Co-Localizes with −SMA Positive Cells

Liver fibrosis was induced using CCl₄ intoxication by oral gavage for 6 weeks in rats pretreated with phenobarbital for 2 weeks. Immunohistochemical analysis revealed an upregulation in −SMA staining and collagen deposition, indicating the successful establishment of liver fibrosis model (Data not shown). In situ hybridization analysis revealed the induction of MFAP4 in fibrotic liver which expression is overlapping with −SMA expression pattern (Data not shown), indicating that −SMA positive cells are the main producers of MFAP4 upon activation throughout the course of fibrogenesis.

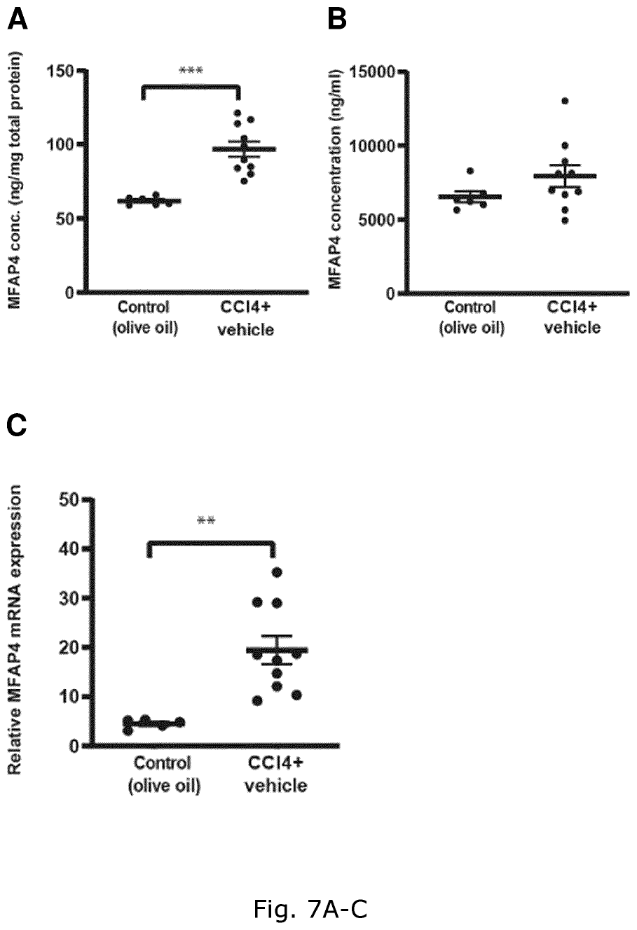

Moreover, MFAP4 level was significantly upregulated in liver homogenate with a corresponding increase in mRNA expression, whereas MFAP4 levels in serum were not significantly upregulated (FIG. 7A-C).

### Antibody-Mediated Targeting of MFAP4 Reduces Gene Expression of Profibrotic Markers.

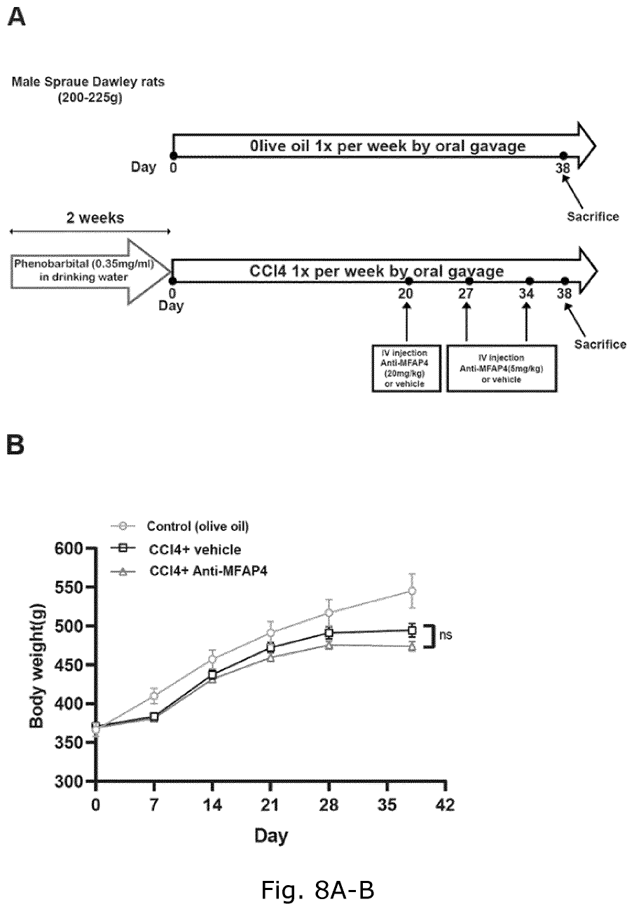

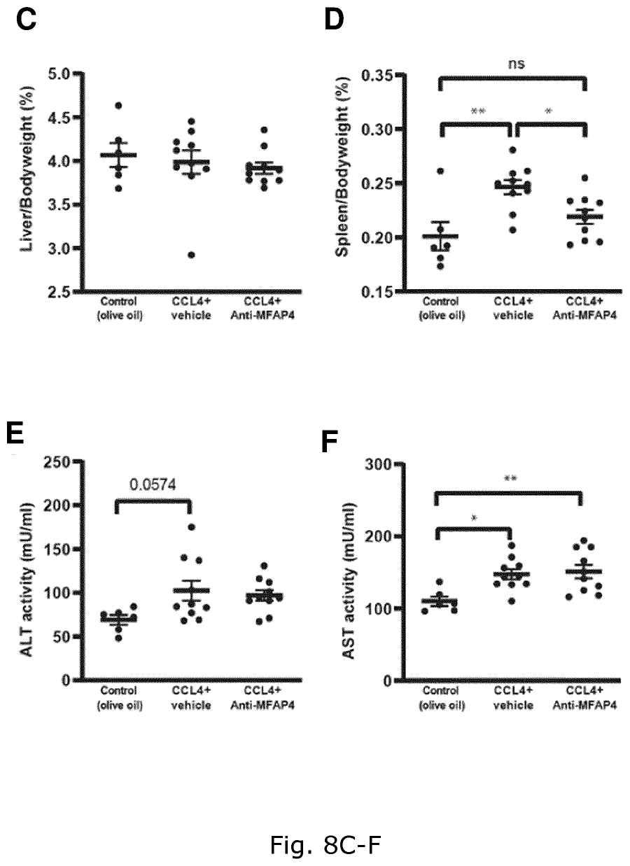

To investigate the effect of MFAP4 neutralization on the progression of liver fibrosis, rats received intravenous injections of anti-MFAP4 (one loading dose of 20 mg/ml and 2 maintenance doses of 5 mg/ml) or vehicle control (histidine/trehalose) on day 20, 27 and 34 after CCl₄ administration (FIG. 8A). Immunohistochemical staining of anti-MFAP4 antibody reveal the same pattern as MFAP4 expression validating the high specificity of anti-MFAP4 antibody for targeting MFAP4 (Data not shown).

Interestingly, a significant decrease in spleen/body weight ratio was observed in the CCl₄+anti-MFAP4 treated group compared to the CCl₄+vehicle group in which the spleen/body weight ratio was significantly induced relative to the control (olive oil) (FIG. 8C-D). However, AST and ALT levels, markers of liver functions, upregulated following CCl₄ induced liver injury were not changed following anti-MFAP4 (FIG. 8E-F).

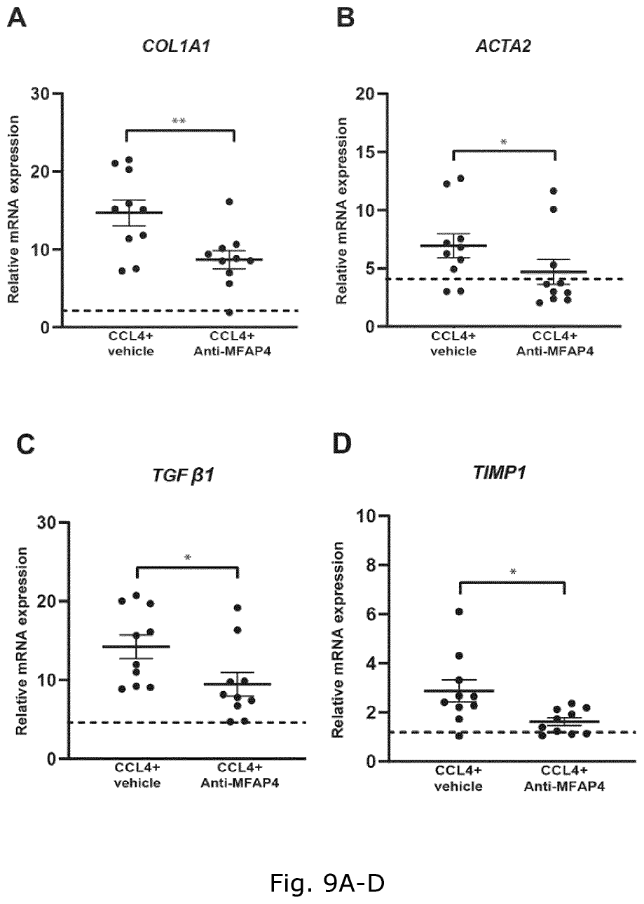

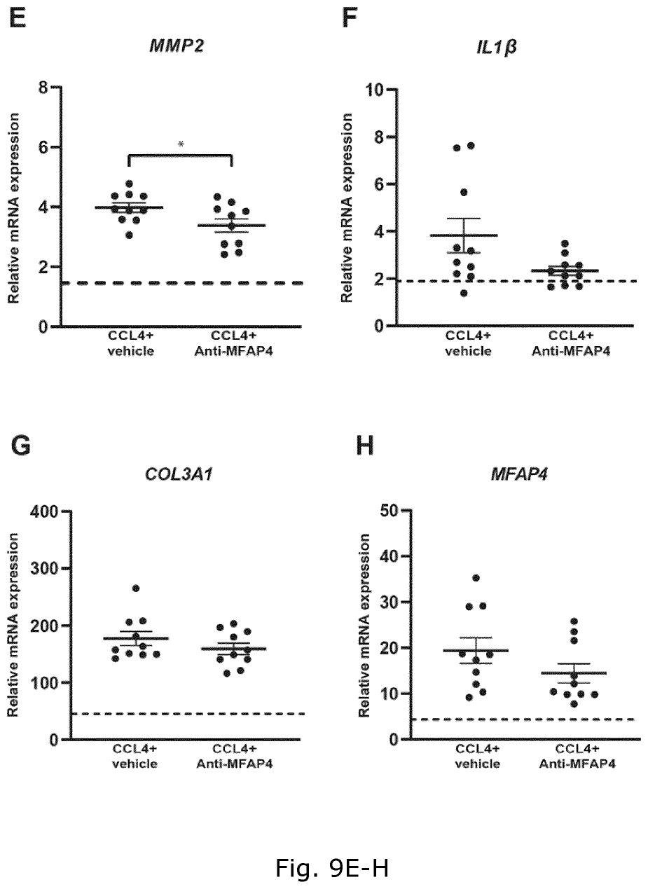

On the other hand, anti-MFAP4 significantly inhibit the CCl₄ induced upregulated gene expression of profibrotic markers, most importantly Col1A1, TIMP1, ACTA2, TGFβ1 and MMP2 and showed a tendency to reduce other profibrotic and proinflammatory markers, IL1β and MFAP4 (FIG. 9A-H).

Blocking MFAP4 Protects from Fibrosis Progression Following CCl₄ Induced Liver Injury

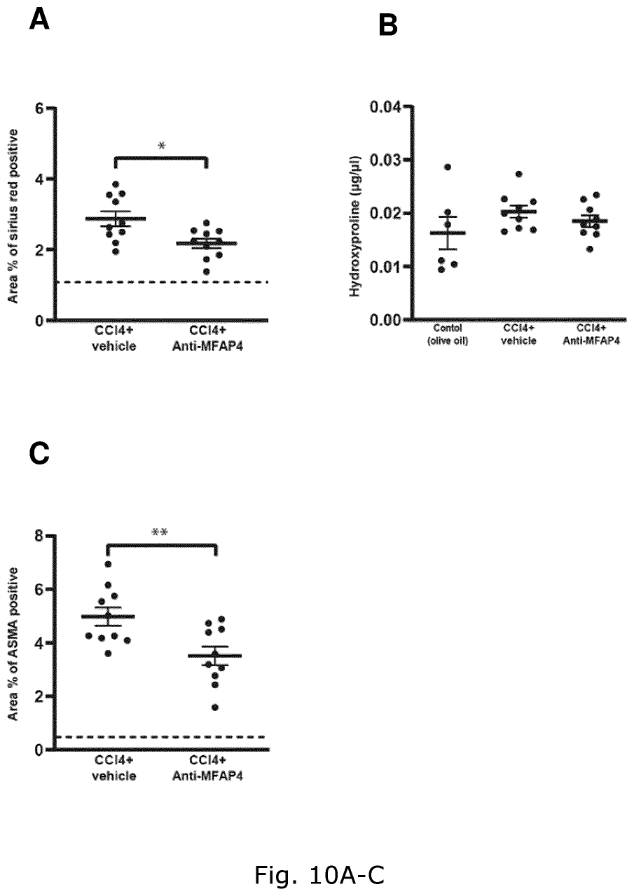

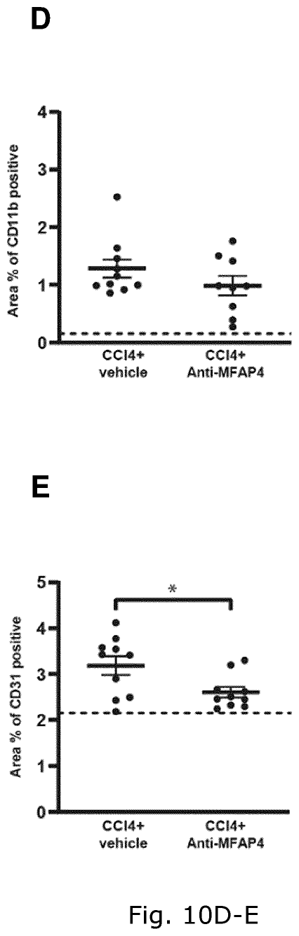

Liver Fibrosis is a complex multistep process, involving a crosstalk between different types of cells and the progression of well characterized events such as inflammation, angiogenesis and most importantly ECM remodeling originating in excessive collagen deposition and fibrosis. In order to understand the mechanism by which MFAP4 is involved in the progression of liver fibrosis, different aspects of the disease were targeted. Consistent with the gene expression results, collagen deposition detected by Sirius red staining (FIG. 10A) as well as −SMA expression (FIG. 10C) was significantly lowered in the antibody treated group compared to the vehicle control indicating reduction in activated myofibroblasts however no difference was detected in the amount of hydroxyproline (FIG. 10B).

Macrophage infiltration was detected by staining for CD11b positive cells. Anti-MFAP4 administration to CCl₄ treated rats showed a tendency towards reduced CD11b positive cells infiltration, which represents the population of activated macrophages, but no significant difference compared to the CCl₄ treated rats receiving vehicle control (FIG. 10D).

Capillarization of liver sinusoid is the hallmark of the disrupted vasculature architecture occurring in liver fibrosis, in which liver sinusoidal endothelial cells (LSEC) loose their fenestrations and acquire a vasculature phenotype with increase in CD31 expression. Quantifying CD31 positive area in the parenchymal regions, revealed a significant decrease in capillarization of sinusoids in CCl₄ treated rats receiving anti-MFAP4 as compared to the CCl₄+vehicle group livers (FIG. 10E).

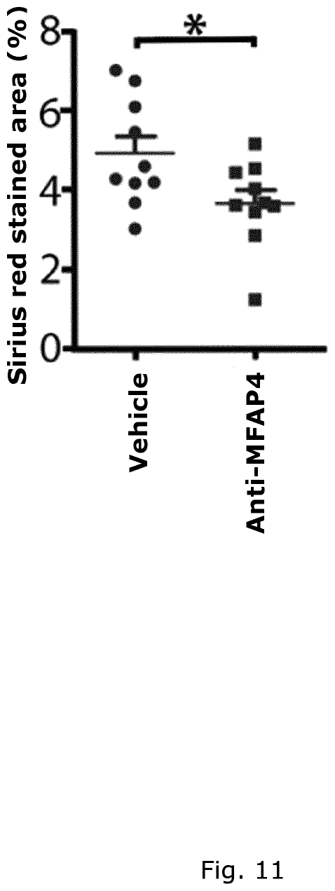

A mouse model was used to validate the anti-fibrotic efficacy of anti-MFAP4 in another species and non-alcoholic fatty liver disease/non-alcoholic steatohepatitis (NAFLD/NASH) model of liver fibrosis. This model supported that intravenous intervention with anti-MFAP4 significantly reduced collagen deposition in the diseased liver (FIG. 11).

### Conclusion

MFAP4 mediates cellular adhesion and enhances PDGF-mediated migration of HHSteCs in vitro. These effects were reversed in the presence of anti-MFAP4 antibody, anti-integrin αvβ3 or RGD peptides. In addition, MFAP4 is synthesized by HHSteCs upon TGFβ1 stimulation and enhances the profibrotic effect of TGFβ1 on HHSteCs in vitro.

Experimental model of liver fibrosis was established in rats after phenobarbital+carbon tetrachloride (CCl₄) intoxication for 8 weeks. Rats received intravenous injections of anti-MFAP4 antibody or vehicle control from week 5 to 8 during CCl₄ administration.

CCl₄+phenobarbital intoxication increased MFAP4 expression in serum and liver of rats. Antibody-mediated blocking of MFAP4 reduced gene expression of profibrotic markers in the liver including Col1A1, Acta2, Tgfβ1, Mmp2 and Timp1. It lowered collagen deposition assessed by quantification of collagen deposition (Sirius red staining), myofibroblast activation assessed by quantification of −SMA immunohistochemical staining, it limits sinusoid capillarization evaluated by CD31 IHC expression in liver parenchymal areas. No significant effect was observed on CD11b expression representing macrophage infiltration. Anti-MFAP4-mediated reduction of deposition of collagen was validated in a mouse model of non-alcoholic fatty liver disease/non-alcoholic steatohepatitis (NAFLD/NASH).

### Example 7—Anti-MFAP4 Inhibits Experimental Chemically Induced Stenotic Anastomosis Formation

### Aim

To study the efficacy of anti-MFAP4 on inhibition of stenotic anastomosis, which may arise as a consequence of surgery-induced fibrosis.

### Materials and Methods

### Study Design

This study is designed as a placebo-controlled trial, including 18 female piglets. In the study two hand sewn anastomoses were created in each piglet on day 1. All anastomoses were injected with 0.8 ml of the sclerotic agent aethoxysclerol (0.05 mg/ml) in 16 predefined subserosal depots.

On day 14 nine piglets received 0.8 ml placebo, and nine piglets received 0.8 ml anti-MFAP4 antibody (hAS0326) in the same predefined subserosal depots. The placebo consisted of 10 mM histidine 10% w/w trehalose pH 6.0 buffer (vehicle). Anti-MFAP4 was administered with a concentration of 40 mg/ml. On postoperative day 28, the anastomoses were extracted and analyzed. Histopathological assessment was performed by a blinded pathologist.

### Animals

A total of 18 weaned female piglets of approximately 20 kg was used. The animals were acclimatized to their new environment for at least one week prior to the trial. They were housed at a conventional large animal housing facility at a constant temperature of 20-21° C. Light/dark cycles: 12 hours with gradually dimmed light and natural light from windows. Access to food: Twice a day (0.9 kg/20 kg body weight) and free access to water. To ensure animal welfare the piglets were intensively inspected daily.

### Anesthesia, Postoperative Treatment and Euthanizing

Pre-anesthetic sedation was a combination of 0.2 mg/kg midazolam (Midazolam Hameln, Hameln Pharma Plus GmbH, Hameln, Germany), 0.04 mg/kg medetomidine (Sedator, Novartis, Copenhagen, Denmark), 0.03 mg/kg buprenorphin (Temgesic, Indivior, Berkshire, England) and 0.05 mg/kg atropin (Atropin, Amgros I/S, Copenhagen, Denmark) administered intramuscularly.

Anesthesia was deepened with 5 mg/kg propofol (B. Braun Medical A/S, Copenhagen, Denmark) intravenously (iv) through an ear vein and intubated with a cuffed tube size 4.0. Anesthesia was maintained with 2.1% isoflurane (Isofluran Baxter, Baxter A/S, Hillerod, Denmark), 1.8% in oxygen/air (1:1) on a MCM 801 ventilator (Dameca, Rødovre, Denmark).

The animals were mechanically ventilated at a respiratory frequency of 16 per minute with a tidal volume of 10 ml/kg. Blood pressure, electrocardiogram, heart rate and oxygen saturation were monitored continuously. Peri-operative analgesia was 50 g/kg/hour iv fentanyl (B. Braun Medical A/S, Copenhagen, Denmark). The piglets received preoperatively prophylactic antibiotics 15 mg/kg amoxicillin (Curamox Prolongatum Vet, Boehringer Ingelheim Danmark A/S, København, Denmark) and perioperatively 20 mg/kg metronidazole (B. Braun, Melsungen AG, Germany), followed by 30 mg/kg amoxicillin (Clamoxyl, Zoetis, Helsinki, Finland) postoperative added to food two times daily for 3 days. Postoperative analgesia consisted of fentanyl transdermal patch 50 g pr. hour. (Pracetam Vet, Ceva Animal Health A/S, Vejle, Denmark). When monitoring the animal postoperatively, buprenorphine (0.125 mg/kg) (Temgesic, Schering-Plough Animal health, New Jersey USA) delivered subcutaneously (s.c.) was administered if there was any sign of pain, and the amount of fentanyl administered was adjusted.

After study completion, the piglets were euthanized using 120 mg/kg pentobarbital (Euthanimal, ScanVet Animal Health A/S, Fredensborg, Danmark) administered via the ear vein.

### Surgical Procedures

The small intestine was exposed through a 10 cm lower midline laparotomy. Two end-to-end anastomoses were performed 50 cm and 150 cm orally to the ileoceacal junction with monocryl 4-0 (Ethicon, Johnson & Johnson, Diegem, Belgium) running suture seromuscular in each piglet. To induce fibrosis a total of 0.8 ml aethoxysclerol 5 mg/ml (Chemische Fabrik Kreussler & Co., Wiesbaden, Germany) was injected in 16 depots proximally and distally to the anastomosis.

The abdominal fascia was closed with running PDS*II® 0 (Ethicon, Johnson & Johnson, Somerville, New Jersey, USA). The skin was closed intracutaneously with a running monocryl® 3-0 (Ethicon, Johnson & Johnson, Diegem, Belgium). The skin incision was sealed with a liquid bandage (KRUUSE Wound Plast, Cat. no: 161020).

A re-laparotomy was performed on postoperative day 14, and the anastomoses were identified and freed from adhesions. The adhesions were graded in accordance to Leach grading of adhesion (Kim et al., 2013). A total of 0.8 ml of placebo or anti-MFAP4 was administered subserosally in 16 depots (0.05 ml) proximal and distal to the anastomosis in 2 mm distance from the anastomotic line. The fascia and the skin were closed as described previously.

On postoperative day 28, the piglets were sedated and subjected to anesthesia as described above. A re-laparotomy was performed, and the anastomoses were identified and freed from adhesions. The adhesions were again graded in accordance to Leach grading of adhesion. All the anastomoses were examined for macroscopic findings including stenosis. The diameter of the bowel was measured at the site of the anastomosis and 10 cm proximally and distally to the anastomosis.

### Radiography

To examine the degree of the anastomotic stricture, the intestinal loop was clamped in vivo approximately 10 cm proximally and distally to each anastomosis. Water-soluble contrast was infused to a pressure of 20 mmHg and an image in one plane was obtained. A control needle of 3 cm was present in every X-ray image to be able to scale the images. X-rays were digitalized and the diameters were measured proximally and distally to the anastomotic level using image analysis software (Image J, NIH, Bethesda, USA).

An anastomotic index (AI) was calculated for each anastomosis by dividing the diameter of the anastomosis multiplied by two by a mean pre- and post-anastomotic diameter based on five measure points within 1 cm intervals from the anastomosis. A ratio of 0.5 is equal to a 50% reduction at the anastomotic site.

### Maximal Anastomotic Tensile Strength (MATS)

The anastomoses were mounted individually in the testing instrument (LF Plus; Lloyds Instruments, Fareham, UK) equipped with a XLC 100N loadcell (Lloyds Instruments, Fareham, UK). The anastomoses were resected with approximately 5 cm margin proximally and distally. The segments were cleaned of fecal contents with water prior to the tensile strength test which was performed 5 minutes after resection to prevent the influence of cold ischemia on the MATS. There was 60 mm between the clamps, with the anastomosis placed in the middle. The segment was stretched with a rate of 15 mm/min until a transmural rupture occurred. The force applied was measured at two different occasions; when a tear became visible in the serosa (MATS-1), and when a transmural rupture appeared (MATS-2).

A simultaneous drop in the load-strain calculated by the software was used to confirm the rupture point of MATS-2. MATS-3 is the maximal force applied during the tensile strength test and was calculated by the software.

### Histopathological Analysis

Tissue samples from the anastomosis were collected for histopathological analysis and histochemical analysis after completion of the tensile-strength test. The distal half of the anastomosis was fixed in a 10% formaldehyde solution for eight days and subsequently embedded in paraffin. The paraffin embedded sections were sliced in 3 m thick sections and stained with sirius red (collagen deposition/fibrosis). Collagen deposition/fibrosis at the anastomotic line was estimated in percentage. Histological analyses were evaluated by an experienced pathologist blinded for the intervention.

### Statistical Analysis

MATS, the anastomotic healing evaluated by histology and the degree of stenosis were compared between groups by non-parametric and parametric tests. Two-tailed Fisher's exact test were used to assess potential group differences in histological variables. Multiple linear regression was used to assess whether injection of anti-MFAP4 or placebo, and histological score had influence on MATS. The results were considered statistically significant if the p-value is under 0.05. Statistical analyses were performed using Stata/BE (version 17; Texas, USA).

### Results

### Maximal Anastomotic Tensile Strength

Five anastomoses from the placebo group were excluded from the tensile strength test. Some were excluded due to software errors during MATS test, and others were excluded due to misinterpretation of transmural rupture. The placebo group thereby included 13 anastomoses and the treatment group included 18 anastomoses.

The treatment group had the highest mean MATS values in all three tests compared to the placebo group and all the strength tests were significant before adjusting for adherences and weight gain. When adjusted only MATS 2 and 3 remained significant (Table 4).

TABLE 4
Maximal Anastomotic Tensile Strength, Anastomotic
Index, Adhesions, Macroscopic Findings and Weight
gain in the treatment and placebo group.

| | Placebo | Anti-MFAP4 | | |
|---|---|---|---|---|
| Variables | Mean ± SD | Mean ± SD | p-value | p-value¹ |
| MATS1 | 4.54 ± 1.53 | 6.24 ± 1.82 | 0.004 | 0.100 |
| MATS2 | 11.97 ± 6.58 | 17.78 ± 4.82 | 0.008 | <0.001 |
| MATS3 | 9.67 ± 6.6 | 18.2 ± 4.68 | 0.005 | <0.001 |
| Anastomotic | 0.65 ± 0.1 | 0.58 ± 0.12 | 0.109 | 0.029 |
| index | | | | |
| Adhesions | 5.33 ± 1.19 | 5.22 ± 0.81 | 0.822 | |
| 1. operation | | | | |
| Adhesions | 4.33 ± 2.52 | 5 ± 1.46 | 0.507 | |
| 2. operation | | | | |
| Diameter 10 cm | 3.39 ± 0.37 | 3.27 ± 0.37 | 0.449 | 0.082 |
| proximal | | | | |
| Diameter at | 3.09 ± 0.39 | 3.06 ± 0.46 | 0.369 | 0.213 |
| anastomotic line | | | | |
| Diameter 10 cm | 3.46 ± 0.43 | 3.32 ± 0.38 | 0.395 | 0.243 |
| distal | | | | |
| Weight gain | 24.14 ± 1.91 | 20.99 ± 3.95 | 0.059 | |

¹Corrected by multiple linear regression.

### Radiography

The anastomotic index was significantly different in the treatment group compared to the control group when adjusted for weight gain and adhesions.

Macroscopic findings

There were no sign of intestinal abscesses, visible leakage, fistulas or signs of ileus. Adhesion scores and difference in the external intestinal diameters proximally, distally or at the anastomotic line were not significantly affected by intervention.

### Collagen Deposition

One anastomosis was excluded due to poor quality of the sample, and it could not be evaluated, leaving 17 anastomoses in the placebo group and 18 in the treatment group. Collagen deposition had a lower mean in the treatment group 19.71±7.49 compared to the placebo group 26.94±15.45 (p-value=0.028).

### Conclusion

The study showed that anti-MFAP4 injections in small intestinal anastomoses in a porcine model has a positive effect in reducing collagen deposition/fibrosis on postoperative day 28. It further increases the maximal tensile strength. Thus, anti-MFAP4 has a positive effect on the relief of induced stenotic anastomosis in the intestine due to fibrosis arisen as a consequence of surgery.

### REFERENCES

Kim, et al. “Comparative study for preventive effects of intra-abdominal adhesion using cyclooxygenase-2 enzyme (COX-2) inhibitor, low molecular weight heparin (LMWH), and synthetic barrier”. Yonsei Med J. 54(6), 1491-7 (2013).
Köhler, G. et al. “Continuous cultures of fused cells secreting antibody of predefined specificity”. Nature 256, 495-497 (1975).
Lassen, N. E. et al. “Coupling of Bone Resorption and Formation in Real Time: New Knowledge Gained From Human Haversian BMUs”. J Bone Miner Res 32, 1395-1405 (2017).
Molleken, C. et al. “Detection of novel biomarkers of liver cirrhosis by proteomic analysis”. Hepatology 49, 1257-1266 (2009).
Rognes, T.: “ParAlign: a parallel sequence alignment algorithm for rapid and sensitive database searches”. Nucleic Acids Research 29(7), 1647-1652 (2001).
Schlosser, A. et al. “MFAP4 Promotes Vascular Smooth Muscle Migration, Proliferation and Accelerates Neointima Formation”. Arterioscler Thromb Vasc Biol 36, 122-133 (2016).
Schlothauer, T. et al. “Novel human IgG1 and IgG4 Fc-engineered antibodies with completely abolished immune effector functions.” Protein Eng Des Sel 29, 457-466 (2016).
Smith, T. F. et al. “Identification of Common Molecular Subsequences”. J. Mol. Biol. 147, 195-197 (1981).
Sækmose, S. G. et al. “Enzyme-Linked Immunosorbent Assay characterization of Basal Variation and Heritability of Systemic Microfibrillar-Associated Protein 4”. PLoS One 8(12), e82383 (2013).
Wulf-Johansson, H. et al. “Localization of Microfibrillar-Associated Protein 4 (MFAP4) in Human Tissues: Clinical Evaluation of Serum MFAP4 and Its Association with Various Cardiovascular Conditions”. PLoS One 8, e82243 (2013).

### SEQUENCE LISTING

SEQ ID NO: 1—light chain variable region (mAS0326)
SEQ ID NO: 2—heavy chain variable region (mAS0326)
SEQ ID NO: 3—light chain variable region (hAS0326)
SEQ ID NO: 4—heavy chain variable region (hAS0326)
SEQ ID NO: 5—light chain variable region Hyb 7-14
SEQ ID NO: 6—heavy chain variable region Hyb 7-14
SEQ ID NO: 7—light chain variable region Hyb 7-5
SEQ ID NO: 8—heavy chain variable region Hyb 7-5
SEQ ID NO: 9—light chain variable region; CDR1
SEQ ID NO: 10—light chain variable region; CDR2
SEQ ID NO: 11—light chain variable region; CDR3
SEQ ID NO: 12—heavy chain variable region; CDR1
SEQ ID NO: 13—heavy chain variable region; CDR2
SEQ ID NO: 14—heavy chain variable region; CDR3
SEQ ID NO: 15—heavy chain hAS0326 Fc-neutralised

### ITEMS

1. A selective targeting agent, which is capable of binding MFAP4 and inhibiting MFAP4-integrin interaction for use in the prevention, alleviation and/or treatment of fibrosis.
2. The selective targeting agent for use according to item 1, wherein fibrosis is liver fibrosis or chronic liver diseases, lung fibrosis, eye fibrosis, kidney fibrosis, heart fibrosis, intestinal fibrosis and/or fibrosis in response to surgery or injury.
3. The selective targeting agent for use according item 2, wherein fibrosis is liver fibrosis or chronic liver diseases.
4. The selective targeting agent for use according to item 3, wherein said chronic liver diseases are cirrhosis, liver failure and portal hypertension.
5. The selective targeting agent for use according to item 2, wherein said fibrosis is eye fibrosis.
6. The selective targeting agent for use according to item 5, wherein eye fibrosis is retinal fibrosis.
7. The selective targeting agent for use according to any of the preceding items, wherein said selective targeting agent is an antibody.
8. The selective targeting agent for use according to item 7, wherein said antibody is selected from the group consisting of polyclonal antibodies, monoclonal antibodies, chimeric antibodies, a fully human antibody, a humanized antibody such as a humanized monoclonal antibody, an antibody wherein the heavy chain and light chain are connected by a flexible linker, single chain antibodies, Fc fragments, Fv fragments, scFv fragments, diabody, triabody, Fab fragments, Fab′ fragments, F(ab′)₂ fragments, conjugates having antibody CDR regions, nanobodies, fusion proteins, and a Fab expression library.
9. The selective targeting agent for use according to any of the items 7-8 comprising
   a light chain variable region comprising the amino acid sequence of SEQ ID NO: 1, 3, 5 or 7, or sequences having at least 80% sequence identity, such as at least at least 90% sequence identity, or such as at least 95% sequence identity to SEQ ID NO: 1, 3, 5, or 7; and
   a heavy chain variable region comprising the amino acid sequence of SEQ ID NO: 2, 4, 6 or 8, or sequences having at least 80% sequence identity, such as at least 90% sequence identity, or such as at least 95% sequence identity to SEQ ID NO: 2, 4, 6, or 8.
10. The selective targeting agent for use according to any of the items 1-6, wherein said selective targeting agent is an antibody mimetic protein and/or an aptamer.
11. The selective targeting agent for use according to item 10, wherein
   said antibody mimetic protein is selected from the group consisting of affibody molecules, affilins, affimers, affitins, alphabodies, anticalins, avimers, DARPins, fynomers, kunitz domain peptides, knottins, monobodies and nanoCLAMPs, and/or
   said aptamer is selected from the group consisting of a DNA aptamer, a RNA aptamer, an XNA aptamer and a peptide aptamer.
12. A composition for use in the prevention, alleviation and/or treatment of fibrosis, wherein said composition comprises a selective targeting agent as described in any one of the items 1-11 and one or more physiologically acceptable carriers, excipients and/or diluents.
13. The selective targeting agent for use according to any of the items 1-11 and/or the composition for use according to item 12, wherein the selective targeting agent and/or the composition is administered orally, parenterally, intravenously, intradermally, subcutaneously, systemically, intra-arterially, intramuscularly, intrathecally, intraocularly, intraconjuctivally, intravitreally, intranasally, by inhalation, topically or by direct or catheter guided local organ administration.
14. The selective targeting agent and/or composition for use according to item 13, wherein the selective targeting agent and/or the composition is administered by intravenous, intravitreally or intraperitoneal injection.
15. The selective targeting agent and/or the composition for use according to any of the items 13-14, wherein the selective targeting agent and/or composition is administered by injection or inhalation into the fibrotic area.

1.-12. (canceled)

13. A method of preventing, alleviating and/or treating fibrosis, said method comprising administering an antibody, which is capable of binding MFAP4 and inhibiting MFAP4-integrin interaction, to a subject.

14. The method according to claim 1, wherein the fibrosis is selected from the group consisting of liver fibrosis, chronic liver diseases, lung fibrosis, eye fibrosis, kidney fibrosis, heart fibrosis, intestinal fibrosis and fibrosis in response to surgery or injury.

15. The method according to claim 1, wherein fibrosis is liver fibrosis or chronic liver diseases.

16. The method according to claim 1, wherein fibrosis is chronic liver diseases and said chronic liver diseases are selected from the group consisting of cirrhosis, liver failure and portal hypertension.

17. The method according to claim 1, wherein said fibrosis is eye fibrosis.

18. The method according to claim 1, wherein said fibrosis is eye fibrosis, and wherein the eye fibrosis is retinal fibrosis.

19. The method according to claim 1, wherein said antibody is selected from the group consisting of: polyclonal antibodies, monoclonal antibodies, chimeric antibodies, a fully human antibody, a humanized antibody, a humanized monoclonal antibody, an antibody wherein the heavy chain and light chain are connected by a flexible linker, single chain antibodies, Fc fragments, Fv fragments, scFv fragments, diabody, triabody, Fab fragments, Fab′ fragments, F(ab′)₂ fragments, conjugates having antibody CDR regions, nanobodies, fusion proteins, and a Fab expression library.

20. The method according to claim 1, wherein the antibody comprises
a light chain variable region comprising the amino acid sequence of SEQ ID NO: 1, 3, 5 or 7, or sequences having at least 80% sequence identity to SEQ ID NO: 1, 3, 5, or 7; and
a heavy chain variable region comprising the amino acid sequence of SEQ ID NO: 2, 4, 6 or 8, or sequences having at least 80% sequence identity to SEQ ID NO: 2, 4, 6, or 8.

21. The method according to claim 1, wherein the antibody comprises
a light chain variable region comprising the amino acid sequence of SEQ ID NO: 1, 3, 5 or 7, or sequences having at least 90% sequence identity to SEQ ID NO: 1, 3, 5, or 7; and
a heavy chain variable region comprising the amino acid sequence of SEQ ID NO: 2, 4, 6 or 8, or sequences having at least 90% sequence identity to SEQ ID NO: 2, 4, 6, or 8.

22. The method according to claim 1, wherein the antibody comprises:
a light chain variable region comprising the amino acid sequence of SEQ ID NO: 1, 3, 5 or 7; and
a heavy chain variable region comprising the amino acid sequence of SEQ ID NO: 2, 4, 6 or 8.

23. The method according to claim 1, wherein the antibody is selected from the group consisting of:
an antibody having a light chain variable region according to SEQ ID NO: 1 and a heavy chain variable region according to SEQ ID NO: 2;
an antibody having a light chain variable region according to SEQ ID NO: 3 and a heavy chain variable region according to SEQ ID NO: 4;
an antibody having a light chain variable region according to SEQ ID NO: 5 and a heavy chain variable region according to SEQ ID NO: 6; and
an antibody having a light chain variable region according to SEQ ID NO: 7 and a heavy chain variable region according to SEQ ID NO: 8.

24. The method according to claim 1, wherein the antibody comprises:
a light chain variable region comprising
a CDR 1 region according to SEQ ID NO: 9 or according to a sequence where at the most three amino acids differ from SEQ ID NO: 9 with the proviso that the amino acid at position 9 is a Tyr; a CDR 2 region according to SEQ ID NO: 10 or according to a sequence where at the most three amino acids differ from SEQ ID NO: 10; and a CDR 3 region according to SEQ ID NO: 11 or according to a sequence where at the most three amino acids differ from SEQ ID NO: 11 with the proviso that the amino acid at position 6 is a Tyr; and
a heavy chain variable region comprising:
a CDR 1 region according SEQ ID NO: 12 or according to a sequence where at the most three amino acids differ from SEQ ID NO: 12 with the proviso that the amino acid at position 3 is a Trp; a CDR 2 region according to SEQ ID NO: 13 or according to a sequence where at the most three amino acids differ from SEQ ID NO: 13; and a CDR 3 region according to SEQ ID NO: 14 or according to a sequence where at the most three amino acids differ from SEQ ID NO: 14 with the proviso that the amino acid at position 1 is a Glu and the amino acid at position 9 is a Trp.

25. The method according to claim 1, wherein the antibody comprises:
a light chain variable region comprising a CDR 1 region according to SEQ ID NO: 9, a CDR 2 region according to SEQ ID NO: 10 and a CDR 3 region according to SEQ ID NO: 11; and
a heavy chain variable region comprising a CDR 1 region according to SEQ ID NO: 12, a CDR 2 region according to SEQ ID NO: 13 and a CDR 3 region according to SEQ ID NO: 14.

26. The method according to claim 1, wherein the antibody comprises:
a light chain variable region comprising the amino acid sequence of SEQ ID NO: 1 or 3, or sequences having at least 90% sequence identity to SEQ ID NO: 1 or 3 with the proviso that the amino acid at position 32 is a Tyr and the amino acid at position 94 is a Tyr; and
a heavy chain variable region comprising the amino acid sequence of SEQ ID NO: 2 or 4, or sequences having at least 90% sequence identity to SEQ ID NO: 2 or 4 with the proviso that the amino acid at position 33 is a Trp, the amino acid at position 99 is a Glu and the amino acid at position 107 is a Trp.

27. The method according to claim 1, wherein the antibody is administered orally, parenterally, intravenously, intradermally, subcutaneously, systemically, intra-arterially, intramuscularly, intrathecally, intraocularly, intraconjuctivally, intravitreally, intranasally, by inhalation, topically or by direct or catheter guided local organ administration.

28. The method according to claim 1, wherein the antibody is administered by intravenous, intravitreal, or intraperitoneal injection.

29. The method according to claim 1, wherein the selective targeting agent is administered by injection or inhalation into the fibrotic area.

30. The method according to claim 1, wherein said subject is a mammal.

31. The method according to claim 1, wherein said subject is a human.

32. A method of preventing, alleviating and/or treating fibrosis, said method comprising:
administering to a subject a composition comprising an antibody, which is capable of binding MFAP4 and inhibiting MFAP4-integrin interaction; and one or more physiologically acceptable carriers, excipients and/or diluents.


SEQUENCE LISTING (15 sequences)

SEQ ID NO: 1 - protein, 107 aa, Artificial Sequence
  Antibody sequence
DVQIIQSPSYLAASPGETITINCRASKSISKYLAWYQERPGKTNKLLIYSGSTLQSGIPSRFSGSGSGTDFTLTISSLEPEDFAMYYCQQHNEYPFTFGAGTKLELK

SEQ ID NO: 2 - protein, 122 aa, Artificial Sequence
  Antibody sequence
QVQLQQPGADLVKPGTSVKLSCKASGFTFTSYWMHWVKQRPGQGLEWIGVIHPNSGNTKYNEKFRSEATLTVDKSSNTAYIQLSSLTSEDSAVYYCAREMWNYGNSWYFDVWGTGTTVTVSS

SEQ ID NO: 3 - protein, 107 aa, Artificial Sequence
  Antibody sequence
DIQMTQSPSSLSASVGDRVTITCRASKSISKYLAWYQQKPGKAPELLIYSGSTLQSGIPARFSGSGSGTEFTLTISSLQSEDFAVYYCQQHNEYPFTFGQGTKLEIK

SEQ ID NO: 4 - protein, 122 aa, Artificial Sequence
  Antibody sequence
QMQLVQSGPEVKKPGTSVKVSCKASGFTFTSYWMHWVRQARGQRLEWIGVIHPNSGNTKYNEKFRSRVTMTTDTSTSTAYMELRSLRSDDTAVYYCAREMWNYGNSWYFDVWGQGTTVTVSS

SEQ ID NO: 5 - protein, 107 aa, Artificial Sequence
  Antibody sequence
DIVLTQSPSYLAASPGETITINCRASKSISKYLAWYQERPGKTNKLLIYSGSTLQSGIPSRFSGSGSGTDFTLTISSLEPEDFAMYYCQQHNEYPFTFGAGTKLELK

SEQ ID NO: 6 - protein, 123 aa, Artificial Sequence
  Antibody sequence
EVKVVESGGGLVQPGGSLRLSCATSGFTFSDFYMEWVRQPPGKRLEWIAASRNKANDYTTEYSASVKGRFIVSRDTSQSILYLQLNALRAEDTAIYYCARNYYDSSYWYFDVWGAGTTVTVSS

SEQ ID NO: 7 - protein, 108 aa, Artificial Sequence
  Antibody sequence
DIVMTQSTALMAASPGEKVTITCSVSSSISSSNLHWYQQKSETSPKSWIYGTSNLASGVPGRFSGSGSGTSYSLTISSVEAEDAATYYCQQWSSYPLTFGGGTKLEIK

SEQ ID NO: 8 - protein, 120 aa, Artificial Sequence
  Antibody sequence
EVQLQQSGPELVKPGASVKLSCKTSGYTFTSYDMNWVKQRPGQGLEWIGWIFPRDGSTKFNEKFKGKATLTVDTSSTTAYMELHSLTSEDSAVYFCARAEIFFDYGFDYWGQGTTLTVSS

SEQ ID NO: 9 - protein, 11 aa, Artificial Sequence
  Antibody sequence
RASKSISKYLA

SEQ ID NO: 10 - protein, 7 aa, Artificial Sequence
  Antibody sequence
SGSTLQS

SEQ ID NO: 11 - protein, 10 aa, Artificial Sequence
  Antibody sequence
QQHNEYPFTF

SEQ ID NO: 12 - protein, 6 aa, Artificial Sequence
  Antibody sequence
SYWMHW

SEQ ID NO: 13 - protein, 17 aa, Artificial Sequence
  Antibody sequence
VIHPNSGNTKYNEKFRS

SEQ ID NO: 14 - protein, 13 aa, Artificial Sequence
  Antibody sequence
EMWNYGNSWYFDV

SEQ ID NO: 15 - protein, 471 aa, Artificial Sequence
  Antibody sequence
MGWSWILLFLLSVTAGVHSQMQLVQSGPEVKKPGTSVKVSCKASGFTFTSYWMHWVRQARGQRLEWIGVIHPNSGNTKYNEKFRSRVTMTTDTSTSTAYMELRSLRSDDTAVYYCAREMWNYGNSWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK